(19) United States
(12) Patent Application Publication
(10) Pub. No.: US 2026/0196355 A1
(43) Pub. Date: Jul. 9, 2026

(54) MANAGEMENT SYSTEM FOR PROACTIVE AND PERSONALIZED TROUBLESHOOTING, OUTREACH AND ENGAGEMENT

(71) Applicant: MEDTRONIC MINIMED, INC., Northridge, CA (US)

(72) Inventors: Kaushal R. JOSHI, Porter Ranch, CA (US); Gene T. KAFKA, Santa Monica, CA (US); Pratik J. AGRAWAL, Stevenson Ranch, CA (US); Amanda KINNISCHTZKE, Woodbury, MN (US); Siddharth ARUNACHALAM, Sherman Village, CA (US); Michael P. STONE, Long Beach, CA (US); Kevin E. VELADO, Northridge, CA (US); Anuj M. PATEL, Porter Ranch, CA (US); Naveen Mathew Nathan SATHIYANATHAN, Everett, MA (US)

(21) Appl. No.: 19/435,057

(22) Filed: Dec. 29, 2025

Related U.S. Application Data
(60) Provisional application No. 63/742,362, filed on Jan. 6, 2025.

(51) Int. Cl. G16H 50/30; G16H 10/60; G16H 20/17

(52) U.S. Cl. CPC G16H 50/30; G16H 10/60; G16H 20/17

(57) ABSTRACT

A medical device data management system comprises one or more data repositories configured to store collected data associated with a patient and one or more medical devices of the patient, one or more insight generation engines configured to generate insights related to the patient's experience associated with the one or more medical devices based on the collected data, an intervention engine configured to determine personalized interventions for the patient based on the insights, and one or more communication interfaces configured to provide the personalized interventions to one or more users of the medical device data management system.

### CROSS-REFERENCE TO RELATED APPLICATION

This application claims the benefit of and priority to U.S. Provisional Application No. 63/742,362, filed Jan. 6, 2025, entitled “MANAGEMENT SYSTEM FOR PROACTIVE AND PERSONALIZED TROUBLESHOOTING, OUTREACH AND ENGAGEMENT,” which is assigned to the assignee hereof and is hereby incorporated by reference in its entirety for all purposes.

### TECHNICAL FIELD

The present disclosure relates generally to a medical device data management system for improving medical device user experience.

### BACKGROUND

Portable medical devices may be useful for patients with conditions that need to be monitored and/or treated on a continuous or frequent basis. For example, individuals with diabetes may use continuous glucose monitoring (CGM) sensors and portable insulin delivery devices to keep their blood glucose (BG) level within a target range. In one example, a blood glucose level management system may be a closed-loop system that may include a pump automatically or semi-automatically controlled based at least in part on measurement results of a CGM sensor to deliver appropriate amounts of insulin to a patient. Various data, such as device data, patient physiological condition data, patient experience data, patient activity data, and the like, can be collected before, during, or after the use of the portable medical devices.

### SUMMARY

This disclosure relates generally to a medical device data management system for improving medical device user experience. More specifically, techniques disclosed herein relate to systems and methods for proactive and personalized intervention, troubleshooting, training, and coaching associated with the use of medical devices, using a combination of machine learning (ML) models and various data collected before, during, and/or after the use of the medical devices. Techniques disclosed herein may be practiced in a variety of ways, such as using a server, a user device, a processor-implemented method, a system comprising one or more processors and one or more processor-readable media, and/or one or more (non-transitory) processor-readable media.

According to certain embodiments, a processor-implemented method may include obtaining data associated with a user and one or more medical devices of the user, generating insights related to the user's experience associated with the one or more medical devices based on the obtained data, determining personalized interventions for the user based on the insights, and providing the personalized interventions through one or more communication channels or user interfaces.

According to certain embodiments, a system may include one or more processors, and one or more processor-readable storage media storing instructions. The instructions, when executed by the one or more processors, may cause performance of operations including obtaining data associated with a user and one or more medical devices of the user, generating insights related to the user's experience associated with the one or more medical devices based on the obtained data, determining personalized interventions for the user based on the insights, and providing the personalized interventions through one or more communication channels or user interfaces.

According to certain embodiments, a medical device data management system may include one or more data repositories configured to store collected data associated with a patient and one or more medical devices of the patient, one or more insight generation engines configured to generate insights related to the patient's experience associated with the one or more medical devices based on the collected data, an intervention engine configured to determine personalized interventions for the patient based on the insights, and one or more communication interfaces configured to provide the personalized interventions to one or more users of the medical device data management system.

This summary is neither intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim. The foregoing, together with other features and examples, will be described in more detail below in the following specification, claims, and accompanying drawings.

### BRIEF DESCRIPTION OF THE DRAWINGS

The above and other aspects and features of the disclosure will become more apparent in view of the following detailed description when taken in conjunction with the accompanying drawings wherein like reference numerals identify like elements.

FIG. 1 illustrates an example of a blood glucose level management system according to certain embodiments.

FIG. 2 is a block diagram of an example of a blood glucose level management system according to certain embodiments.

FIG. 3 includes a block diagram illustrating an example of a medical device data management system according to certain embodiments.

FIG. 4 illustrates an example of a process of medical device data management according to certain embodiments.

FIG. 5 illustrates a simplified example of an implementation of a medical device data management system disclosed herein according to certain embodiments.

FIG. 6 illustrates an example of a machine learning process that may be used to train the machine learning models used in the medical device data management system disclosed herein according to certain embodiments.

FIG. 7 includes a flowchart illustrating an example of a processor-implemented method for medical device data management according to certain embodiments.

FIG. 8 is a block diagram of an example of a computer system that can be utilized to implement some embodiments described herein.

The figures depict embodiments of the present disclosure for purposes of illustration only. One skilled in the art will readily recognize from the following description that alternative embodiments of the structures and methods illustrated may be employed without departing from the principles, or benefits touted, of this disclosure.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Techniques disclosed herein relate generally to generally to a medical device data management system. More specifically, techniques disclosed herein relate to systems and methods for proactive and personalized intervention, troubleshooting, training, and coaching associated with the use of medical devices, using a combination of machine learning (ML) models various data collected before, during, and/or after the use of the medical devices. The medical device data management system disclosed herein may be used to provide better support to medical device users, thereby improving the user experience and therapeutical outcomes.

Portable medical devices may be used by patients for condition monitoring and/or treatment on a continuous or frequent basis. For example, diabetes mellitus is a disease of the glucose regulatory system of a patient, where the naturally produced insulin in the body may not be sufficient to control the glucose level in the blood stream of the patient, due to insufficient production of insulin and/or insulin resistance, and thus a diabetic patient may need to receive insulin from a pump or another delivery device based on the patient's physiological condition (e.g., glucose level) and activities (e.g., meal intake) to control the glucose level in the patient's blood stream. To control the glucose level, a diabetic patient's therapy routine may generally include dosages of basal insulin and bolus insulin. Basal insulin, also referred to as background insulin, may include continuous or constant release of small amounts of insulin to keep blood glucose levels at consistent levels during long time periods. Bolus insulin may be taken specifically before, at, or after mealtimes or other times where there may be a rapid increase in the blood glucose level. The dose of insulin to be delivered may be determined based on, for example, the carbohydrate count of a meal and/or the glucose levels of the patient measured using a glucose monitor, such as a fingerstick blood glucose measurement device or a continuous glucose monitoring (CGM) sensor. In one example, to counteract an increase in a patient's blood glucose level resultant from the consumption of a meal (or drink), insulin of a certain dose (referred to as a meal bolus) may be delivered to the patient prior to, contemporaneously with, or shortly after the start of the meal. The dose of the insulin may be determined using an insulin calculator (e.g., an App on a user device) that may consider factors such as the intake of carbohydrates, insulin sensitivity factor (ISF) of the patient, the patient's physiological condition (including the current glucose level), the target glucose range, and the like, and may indicate the number of units of insulin to be delivered. Too much insulin can lead to hypoglycemia, while too little insulin can lead to hyperglycemia.

A patient's experience of using a medical device may not only be affected by the performance of the medical device (e.g., accuracy, reliability, outcome, and lifetime of the medical device), but may also be affected by factors such as the ordering and shipping process, training or guidance provided to users, technical support or customer service (e.g., troubleshooting) provided to users, real-time alerts, alarms, warnings, reminders, tips, or other messages provided to users, and the like. In many cases, relevant data may be lacking or may not be readily available to users. In some cases, raw data related to a patient and the medical device used by the patient may be available, but it can be difficult or time-consuming to understand the data and obtain useful information from the data. For example, a customer service or support team member may not know the preferences of a patient, history of using the medical device by the patient, past issues or complaints from the patient, information associated with patient (e.g., the patient's health condition, age, lifestyle, etc.), or other factors that may affect the experience of a particular patient using a medical device, and thus may not be able to provide prompt, effective support, instructions, and other information that a patient may need to use a medical device, which may negatively impact the patient experience and harm the brand reputation. Without relevant data or meaningful insights extracted from the data, it may also be difficult to provide proactive support to patients or perform precision troubleshooting for the patients.

According to certain embodiments disclosed herein, in order to provide users with better user experience, better support, and better therapeutical results, various data associated with a patient's use of one or more medical devices may be collected before, during, and/or after the use of the medical devices, and the collected data may be used to provide insights, training, customer support, troubleshooting, coaching, guidance, and the like, to the user. For example, data related to the user onboarding process, the initial setup of a medical device, user feedback, user interactions with the support or customer service team, user interactions with health care providers, user activities, user meal intakes, user glucose levels, insulin delivery information (e.g., basal and bolus insulin), therapeutical results, alerts or notifications, and the like, may be collected and stored in a data repository. The collected data may then be used to generate insights or other information related to the use of a medical device, such as user experience scores, leading user experience indicators, patient categories, insight prioritization, patient preferences, and the like. In some embodiments, the insights may be fed into a large language model (LLM) that is trained to, for example, generate personalized recommendations for proactive interventions to improve user experience, suggest precision troubleshooting procedures, create customized educational content and training program for users, provide motivational messages and coaching instructions, and the like. The medical device data management system may provide a centralized interface for support teams to view insights and recommendations, may automatically deliver personalized insights and interventions through emails, text messages, web apps, chatbots, or other user preferred communication channels, and may also provide data-driven insights and recommendations to health care providers or caregivers through emails, text messages, web apps, chatbots, and the like.

The medical device management system disclosed herein can leverage patient and device data to predict patient experience score and leading patient experience indicators, and deliver interventions using an omnichannel of a unified platform, to provide personalized troubleshooting and engagement. The predicted patient experience score may highlight individual successes and identify opportunities to improve user experience with the medical devices. The system may also identify patient segmentations of specific users, identify patient goals associated with their therapy, communicate with patients through their preferred methods, recognize patient outcomes, and highlight strengths and opportunities to improve user experience through personalized educational tips and resources. The system may serve to connect with patients empathetically, build trust, and provide meaningful information and comprehensive patient support during all phases of operating the medical devices, thereby improving user success rate, building user confidence in the therapy, and improving user engagement and loyalty. Techniques disclosed herein may also reduce the cost of operation by, for example, using machine learning models to analyze patient data and generate insights, and using the insights and large language models to provide comprehensive patient support and effective patient training related to medical device operation and maintenance. Although not limited to any particular example, the end users of the medical device data management system described herein may include patients, medical device manufacturer/company representatives, healthcare professionals, caregivers, patient coaches, and the like. In some examples, the system described herein may be utilized to provide some or all of the support and interventions with patients if so desired.

Techniques disclosed herein may be implemented on a server, a computer, a user device (e.g., a smartphone), a medical device (e.g., a CGM sensor or an insulin delivery device), and the like, or a combination thereof. In one example, a user app implementing some techniques disclosed herein may be executed on the user device to provide a user with the comprehensive and timely support, thereby improving user experience and therapeutical results. In some examples, the medical device data management system disclosed herein may be implemented using a cloud-based architecture where most of the processing tasks may be performed by one or more remote server systems that communicate with user devices or medical devices, such as cloud-based server systems. The medical device data management system may communicate with many user devices or medical devices and thus can obtain and process patient data for a population of different patients. Patient data can originate from various sources, including insulin infusion devices, continuous glucose sensor devices, mobile client devices, patient owned or operated computer systems, activity tracker devices, navigation or global positioning system (GPS) devices, and the like. The patient data can be stored in data repositories and may be processed and analyzed to generate therapy-related data (e.g., insulin delivery data and glucose level data) and patient engagement data (e.g., user experience/satisfaction data) that can be displayed on a website and/or accessed via a web-based application.

In some examples, the medical device management system disclosed herein may be implemented using a website and an online (e.g., web browser based) application to provide insights, instructions, suggestions, recommendations, advice, patient status or progress reports, and/or guidance to patients, a medical device company representative or support personnel, a caregiver, a healthcare professional, and the like, for achieving proper medical device usage, better therapeutical outcomes, and better patient experience and behavior. The web-based system may provide end users with a variety of interactive web pages that include graphical elements, menus, text entry fields, search fields, output/results screens, and the like. For example, the web-based tool may include, for example, output pages or displays that allow users to review the clinical outcomes of patients, recommendation pages or displays that provide tips, guidance, and suggestions, and the like.

As used herein, an “outcome,” a “therapeutical outcome,” or a “therapeutical result” may refer to a patient-related result associated with medical treatment or therapy. In one example, a “glycemic outcome” is a patient-related result that is associated with the patient's glycemic state, diabetes therapy, insulin status, condition of the insulin infusion device, or the like. For example, a glycemic outcome may correspond to a status of a patient's blood glucose levels, such as within range, high (e.g., hyperglycemic), low (e.g., hypoglycemic), variable, and the like, or may correspond to other metrics that may be indicative of glycemic health, such as glucose management indicator (GMI) values or A1C values, time-in-range values, time in a tight glucose range (TITR) values, and the like.

As used herein, an “insight” may refer to meaningful information, knowledge, or understanding of a subject (e.g., an object, person, event, action, phenomenon, etc.) that is extracted, derived, or otherwise generated from raw data associated with the subject. In some examples, an insight may be statistically derived or may be generated using machine learning techniques. In some examples, an insight may be an association between an action/event (or a collection of actions/events) and a corresponding outcome, or an observation of a particular pattern of interest. For example, an insight may include a predicted patient experience score, a relationship between a patient experience score and actions/events/outcomes associated with a patient and one or more medical devices used by the patient, an estimation of an effect or impact an event/action/outcome may have on the patient experience score, and the like. In one example, an insight may be an association between an action/event (or a collection of actions/events) and a corresponding glycemic outcome as measured by a glucose sensor. An insight generation engine for determining an insight of a subject may be capable of data integration, analysis, discovery, and/or visualization. In some examples, an insight generation engine may apply artificial intelligence (AI) and machine learning (including natural language processing) techniques to analyze, interpret, and extract meaningful information from raw data, and may also provide comprehensive, intuitive, well-organized, and relevant information to users so that users may make more informed, strategic, and swift decisions or action. In certain embodiments, a cloud-based system may be used to implement techniques disclosed herein. The cloud-based system may include one or more servers and data repositories, and may apply a plurality of insight generation techniques (e.g., a plurality of ML models) to patient data and/or device data. There can be any number of meaningful insights and any number of independently executing insight generation techniques or models.

In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, operations, and techniques may be shown without necessary detail or may not be shown, in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as “example” is not necessarily to be construed as preferred or advantageous over other embodiments or designs.

Carbohydrates in food intake may be broken down into glucose (sugar) in the stomach and/or intestine, and may be absorbed at the small intestine and/or large intestine into the blood stream. The blood stream may transport glucose to the capillaries of the body, where some glucose may diffuse into the interstitial fluid between cells (e.g., fat or muscle cells), which may use the glucose for energy. The endocrine system of the human body that directs and regulates the functions and activities of the body (working with the nervous system) may secrete chemicals that transmit messages to tissues and organs. For example, endocrine glands or organs may release hormones into the blood stream for transportation to target cells with receptors. In one example, insulin may be made by the B-cells of pancreas, and may, for example, increase the glucose uptake, enhance glucose utilization, stop hepatic glucose production, stimulate glycogen formation in liver and skeletal muscle, promote protein synthesis, and increase fat storage.

Insulin may function as a key that can unlock cells and help glucose to move into cells where the glucose may be used for energy. Without insulin, glucose may not be able to enter cells to be used for energy, and may build up in the interstitial fluid and blood stream. A healthy pancreas may continuously release a small amount of regular human insulin 24 hours a day, including between meals and during sleep. The small amount of insulin may match the liver's release of glucose. A healthy pancreas may also secrete a larger amount of insulin after food intake to match the amount of food intake.

The liver of a person may absorb excessive glucose from digestion and convert excessive glucose to glycogen for storage so that glucose may be released back to the blood stream when needed. For example, when the blood glucose level is low, the α-cells of the pancreas may secrete glucagon. Glucagon may cause the liver to release the stored form of glucose (glycogen) into the blood stream to help increase the glucose level. The balance between insulin secreted by the β-cells and the glucagon secreted by the α-cells may help to maintain the normal blood glucose level, such as in the range of about 80-140 mg/dL before meals.

The naturally produced insulin in the body of a diabetic patient may not be sufficient to control the glucose level in the blood stream of the patient, due to insufficient production of insulin and/or insulin resistance. Therefore, a diabetic patient may need to receive insulin from a pump or another delivery device such as an injection or infusion device to control the glucose level in the patient's blood stream. To control the glucose level, a diabetic patient's therapy routine may generally include dosages of basal insulin and bolus insulin. Dosages of insulin to be delivered may be determined based on, for example, the carbohydrate count of a meal, and/or the glucose level of the patient measured using a glucose monitor, such as a continuous glucose monitor (CGM).

In some implementations (e.g., in a closed-loop system), the insulin delivery device may communicate with or otherwise use a sensor device (including but not limited to a CGM) to perform various measurements for a patient. In one example, the sensor device may include subcutaneous implanted electrodes to concurrently monitor the patient's response to meals and insulin introduced by the insulin delivery device. The sensor device and the insulin delivery device may be in a communication network (wired or wireless) with one or more processors and/or patient devices (such as a patient's smartphone equipped with an application or other software) to create an overall system for monitoring a patient disease state and for facilitating treatment thereof.

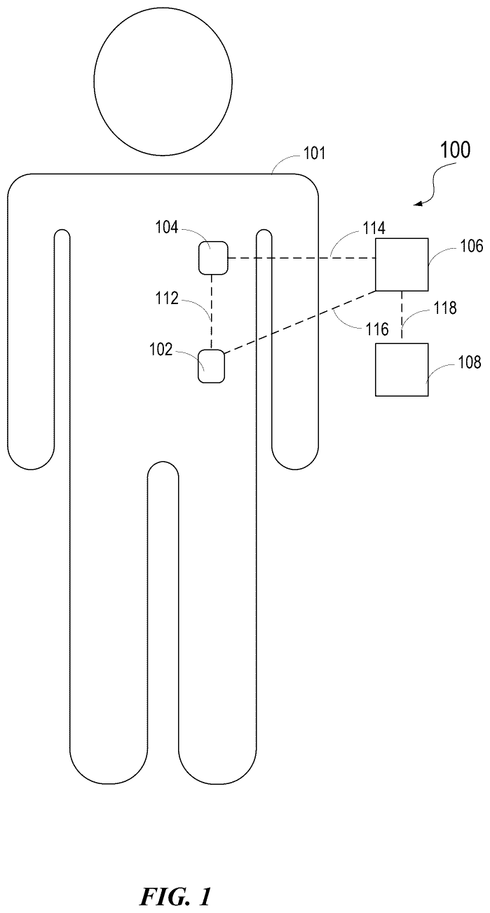

FIG. 1 illustrates an example of a blood glucose level management system 100 according to certain embodiments. Blood glucose level management system 100 may be used to monitor and regulate the blood glucose level of a patient 101. In the illustrated example, blood glucose level management system 100 may include a delivery device 102, a monitoring device 104, a computing device 106, and an optional remote/cloud computing system 108. Delivery device 102, monitoring device 104, and computing device 106 may be embodied in various ways, including being disposed in one or more device housings. For example, in some embodiments, all of devices 102-106 may be disposed in a single device housing. In some embodiments, each of devices 102-106 may be disposed in a separate device housing. In some embodiments, two or more of devices 102-106 may be disposed in the same device housing. In some embodiments, a single device 102, 104, or 106 may have two or more parts that are disposed in two or more housings. For example, monitoring device 104 may include an on-body part and a display and control part communicated with the on-body part through wires or wirelessly. Delivery device 102 may include an on-body site (e.g., including a cannula) and a part that includes a reservoir, a pump, and a control unit. These and other embodiments, and combinations thereof, are contemplated to be within the scope of the present disclosure.

Blood glucose level management system 100 may include a plurality of communication links, such as communication links 112-118. Communications links 112-118 may each be a wired connection and/or a wireless connection. In embodiments where two devices are located in a same housing, the communication link may include, for example, wires, cables, and/or communication buses on a printed circuit board, among other things. In embodiments where two devices are separate from each other in different device housings, the communication links may be wired and/or wireless connections. Wired connections may include, for example, an Ethernet connection, a Universal Serial Bus (USB) connection, and/or another type of physical connection. Wireless connections may include, for example, a cellular connection, a Wi-Fi connection, a Bluetooth® connection, a mesh network connection, and/or another type of connection using a wireless communication protocol. Some embodiments of communication links 112-118 may use direct connections, such as Bluetooth® connections, and/or may use connections that route through one or more networks or network devices (not shown), such as an Ethernet network, a Wi-Fi network, a cellular network, a satellite network, an intranet, an extranet, the Internet, and/or the Internet backbone, among other types of networks. Various combinations of wired and/or wireless connections may be used for communication links 112-118.

Delivery device 102 may be configured to deliver a therapeutic substance to patient 101. The therapeutic substance may include, for example, insulin, HIV drugs, drugs to treat pulmonary hypertension, iron chelation drugs, pain medications, anti-cancer treatments, medications, vitamins, hormones, a nutritional supplement, a dye, a tracing medium, a saline medium, a hydration medium, and the like. Delivery device 102 may be secured to patient 101 (e.g., to the body or clothing of patient 101) or may be at least partially implanted in the body of patient 101. In some embodiments, the delivery device 102 may include a reservoir, an actuator, a delivery mechanism, and a cannula (not shown). The reservoir may be configured to store an amount of the therapeutic substance. In some embodiments, the reservoir may be refillable or replaceable. The actuator may be configured to drive the delivery mechanism. In some examples, the actuator may include a motor, such as an electric motor. The delivery mechanism may be configured to move the therapeutic substance from the reservoir through the cannula. In some examples, the delivery mechanism may include a pump and/or a plunger. The cannula may facilitate a fluidic connection between the reservoir and the body of patient 101. The cannula and/or a needle may facilitate delivery of the therapeutic substance to a tissue layer, vein, interstitial fluid, or body cavity of patient 101. During operation, the actuator, in response to a signal (e.g., a command signal), may drive the delivery mechanism, thereby causing the therapeutic substance to move from the reservoir, through the cannula, and into the body of patient 101.

The components of delivery device 102 described above are merely provided as an example. Delivery device 102 may include other components, such as, without limitation, a power supply, a communication transceiver, one or more processors or other computing resources, memory devices, and/or user interfaces (e.g., buttons, keys, display, etc.), among other things. In some implementations, delivery device 102 may host an App (e.g., an insulin calculator) that may calculate the desired amount of therapeutic substance to be delivered to patient 101. Persons skilled in the art will recognize various implementations of delivery device 102 and the components of such implementations. All such implementations and components are contemplated to be within the scope of the present disclosure.

Monitoring device 104 may be configured to detect a physiological condition (e.g., a glucose concentration level) of patient 101 and may also be configured to detect other physiological conditions. Monitoring device 104 may be secured to the body of patient 101 (e.g., to the skin of patient 101 via an adhesive) and/or may be at least partially implanted into the body of patient 101. Depending on the particular location or configuration, monitoring device 104 may be in contact with biological matter (e.g., interstitial fluid and/or blood) of patient 101.

Monitoring device 104 may include one or more sensors (not shown), such as, without limitation, electrochemical sensors, electrical sensors, and/or optical sensors. As persons skilled in the art will understand, an electrochemical sensor may be configured to respond to the interaction or binding of a biological marker to electrodes by generating an electrical signal based on, for example, a potential, conductance, current, and/or impedance of an electrical path through the electrodes. The electrodes may include a material selected to interact with a particular biomarker, such as glucose. The potential, current, conductance, and/or impedance may correlate with a concentration of the particular biomarker. In one example, the electrochemical sensor may include a glucose limiting membrane (GLM) that limits the amount of glucose and oxygen delivered to a glucose oxidase (GOx) layer of a working electrode of the sensor to ensure that the reactions are glucose limited. The GOx layer or another active enzyme layer on the working electrode of the sensor may break down glucose and oxygen into gluconic acid and hydrogen peroxide. The generated peroxide molecules may interact with the working electrode to break down hydrogen peroxide into two hydrogen ions, oxygen, and two electrons at the surface of the working electrode, when a voltage signal is supplied to the working electrode. The electrical charges may be forced to move between electrodes (e.g., between the working electrode and counter electrode), thereby generating a sensor current signal (Isig) that can be measured by sensor electronics. Other signals such as the counter voltage (Vontr, the voltage potential difference between the counter electrode and the working electrode), electrochemical impedance spectroscopy (EIS) at different frequencies, and the like, may also be measured. The signals measured using the sensor, including the Isig, Ventr, and EIS, may be processed (e.g., filtered or transformed) to generate some other signals or parameters, such as filtered Isig signals, real and imaginary impedance at various frequencies, and the like. These signals and/or the processed parameters may be used in one or more sensor glucose (SG) models (e.g., machine learning models or mathematical models) to determine SG values that may be estimations of the blood glucose (BG) levels of the patient.

As persons skilled in the art would understand, an electrical sensor may be configured to respond to an electrical biosignal by generating an electrical signal based on an amplitude, frequency, and/or phase of the electrical biosignal. The electrical biosignal may include a change in electric current produced by the sum of an electrical potential difference across a tissue, such as the nervous system, of patient 101. In some embodiments, the electrical biosignal may include portions of a potential change produced by the heart of patient 101 over time (e.g., recorded as an electrocardiogram) that may be indicative of a glucose level of patient 101. An optical sensor may be configured to, for example, respond to the interaction or binding of a biological marker to a substrate by generating an electrical signal based on change in luminance of the substrate. In one example, the substrate may include a material selected to fluoresce in response to contact with a selected biomarker, such as glucose. The fluorescence may be proportional to a concentration of the selected biomarker.

In some embodiments, monitoring device 104 may include other types of sensors that may be worn, carried, or coupled to patient 101 to measure activity of patient 101 that may influence the glucose levels or glycemic response of patient 101. As an example, the sensors may include an acceleration sensor configured to detect an acceleration of patient 101 or a portion of the patient 101, such as the person's hands or feet, the position changes of which may be associated with an activity of patient 101. For example, the acceleration or movement (or lack thereof) of the body portion of patient 101 may be indicative of exercise, sleep, or food/beverage consumption activity of patient 101, which may influence the glycemic response of patient 101. In some embodiments, the sensors may measure heart rate and/or body temperature, which may indicate an amount of physical exertion experienced by patient 101. In some embodiments, the sensors may include a Global Positioning System (GPS) receiver which may detect GPS signals to determine a location of patient 101.

The sensors described above are merely provided as examples. Other sensors or types of sensors for monitoring physiological condition, activity, and/or location, among other things, will be recognized by persons skilled in the art and are contemplated to be within the scope of the present disclosure. For any sensor, the signal provided by a sensor may be referred to herein as a “sensor signal.” As used herein, the term “sensed data” may mean and include the information represented by a sensor signal or by a pre-processed sensor signal. In some embodiments, sensed data may include glucose levels of patient 101, acceleration of a part of patient 101, heart rate of patient 101, temperature of patient 101, and/or geolocation (e.g., GPS location) of patient 101, among other things. Monitoring device 104 may communicate sensed data to delivery device 102 via communication link 112 and/or to computing device 106 via communication link 114. Use of sensed data by delivery device 102 and/or by computing device 106 is described in more detail below.

In some embodiments, monitoring device 104 may include components and/or circuitry configured to pre-process sensor signals. Pre-processing may include, for example, amplification, filtering, attenuation, scaling, isolation, normalization, transformation, sampling, and/or analog-to-digital conversion, among other things. In some embodiments, monitoring device 104 may host an App for process the sensor signals. In some embodiments, monitoring device 104 may include a wired or wireless transceiver as described above for transmitting the sensor signals or receiving commands or instructions. Persons skilled in the art will recognize various implementations for such pre-processing, including, without limitation, implementations using processors, controllers, integrated circuits, application specific integrated circuits (ASICs), hardware, firmware, programmable logic devices, and/or machine-executable instructions, among others. The types of pre-processing and their implementations are merely provided as examples. Other types of pre-processing and implementations are contemplated to be within the scope of the present disclosure. In some embodiments, monitoring device 104 may not perform pre-processing.

Computing device 106 may provide processing capabilities and may be implemented in various ways. In some embodiments, computing device 106 may be a consumer device, such as a smartphone, a computerized wearable device (e.g., a smartwatch), a tablet computer, a laptop computer, or a desktop computer, among others, or may be a special purpose device (e.g., a portable control device) provided by, for example, the manufacturer of delivery device 102. In some embodiments, computing device 106 may be processing circuitry that may be integrated with another device, such as delivery device 102. In some embodiments, computing device 106 may be secured to patient 101 (e.g., to the body or clothing of patient 101), may be at least partially implanted into the body of patient 101, and/or may be held by patient 101.

For each of the embodiments of computing device 106, computing device 106 may include various types of logic circuitry, including, but not limited to, microprocessors, controllers, digital signal processors (DSPs), application specific integrated circuits (ASICs), field-programmable gate arrays (FPGAs), central processing units (CPU), graphics processing units (GPU), programmable logic devices, memory (e.g., random access memory, volatile memory, non-volatile memory, etc.), or other discrete or integrated logic circuitry, as well as combinations of such components. The term “processing circuitry” may generally refer to any of the foregoing logic circuitry, alone or in combination with other logic circuitry, or any other circuitry for performing computations.

Some aspects of delivery device 102, monitoring device 104, and computing device 106 have been described above. One or more of devices 102-106 may include a user interface (not shown) that presents information to patient 101 and/or receives information from patient 101. The user interface may include a graphical user interface (GUI), a display device, a keyboard, a touchscreen, a speaker, a microphone, a vibration motor, buttons, switches, and/or other types of user interfaces. Persons skilled in the art will recognize various types of user interfaces that may be used, and all such user interfaces are contemplated to be within the scope of the present disclosure. For example, where computing device 106 is a consumer device such as a smart phone, tablet computer, laptop computer, or the like, the user interfaces would include a display device, a physical and/or virtual keyboard, and/or audio speakers provided by such consumer devices, among other things. In some embodiments, a user interface may notify patient 101 of sensed data (e.g., glucose level) and/or insulin delivery data (e.g., rates of historic, current, or future insulin delivery) and may present alerts to patient 101. In some embodiments, a user interface may receive inputs from patient 101, which may include, for example, a requested change in insulin delivery setting and/or a meal indication, among other things. The descriptions and embodiments above regarding user interfaces are merely provided as examples, and other types and other uses of user interfaces are contemplated to be within the scope of the present disclosure.

In one specific example, the communications between devices 102-106 and cooperation between devices 102-106 may be used for insulin delivery. As depicted in FIG. 1 and as described above, devices 102-106 may communicate with each other via communication links 112-116. In some embodiments, computing device 106 may control operations of delivery device 102 and/or monitoring device 104. For example, computing device 106 may generate one or more signals (e.g., a command signal) that cause delivery device 102 to deliver insulin to patient 101, for example, as a basal dosage and/or a bolus dosage. In some embodiments, computing device 106 may receive data associated with insulin delivery (e.g., insulin delivery data) from delivery device 102 and/or receive sensed data (e.g., glucose levels) from monitoring device 104, and may perform computations based on the insulin delivery data, the sensed data, and/or other data to control delivery device 102. Insulin delivery data may include, but is not limited to, the type of insulin being delivered, historical insulin delivery rates and/or amounts, current insulin delivery rate and/or amount, insulin delivery time, and/or user inputs affecting insulin delivery. As persons skilled in the art will understand, in a closed-loop operating mode, computing device 106 may communicate dosage commands to delivery device 102 based on, for example, a difference between a current glucose level in the body of patient 101 (e.g., received from monitoring device 104) and a target glucose level (e.g., determined by computing device 106 or set on delivery device 102). The dosage commands may indicate an amount of insulin to be delivered and/or a rate (or time) of insulin delivery, and may regulate the current glucose level toward the target glucose level.

Remote/cloud computing system 108 may be any proprietary remote/cloud computing system or a commercial cloud computing system including one or more server computing devices. Remote/cloud computing system 108 may provide alternative or additional computing resources as needed when the computing resources of a client computing device (e.g., computing device 106) are not sufficient. Computing device 106 and remote/cloud computing system 108 may communicate with each other through a communication link 118, which may traverse one or more communication networks (not shown). The communication networks may include, for example, an Ethernet network, a Wi-Fi network, a cellular network, a satellite network, an intranet, an extranet, the Internet, and/or the Internet backbone, among other types of networks. Persons skilled in the art will recognize implementations for remote/cloud computing system 108 and how to interface with such systems through various types of networks. For example, remote/cloud computing system 108 may include an array of processing circuitry and may execute machine-readable instructions. Such implementations, interfaces, and networks are contemplated to be within the scope of the present disclosure.

In some embodiments, remote/cloud computing system 108 may make a therapy determination (e.g., an insulin amount or adjusted insulin amount), and may communicate the therapy to delivery device 102 via computing device 106. In some embodiments, computing device 106 may make the therapy determination and communicate it to delivery device 102. In some embodiments, monitoring device 104 may make the therapy determination and communicate it to delivery device 102 either directly or through an intermediary such as computing device 106.

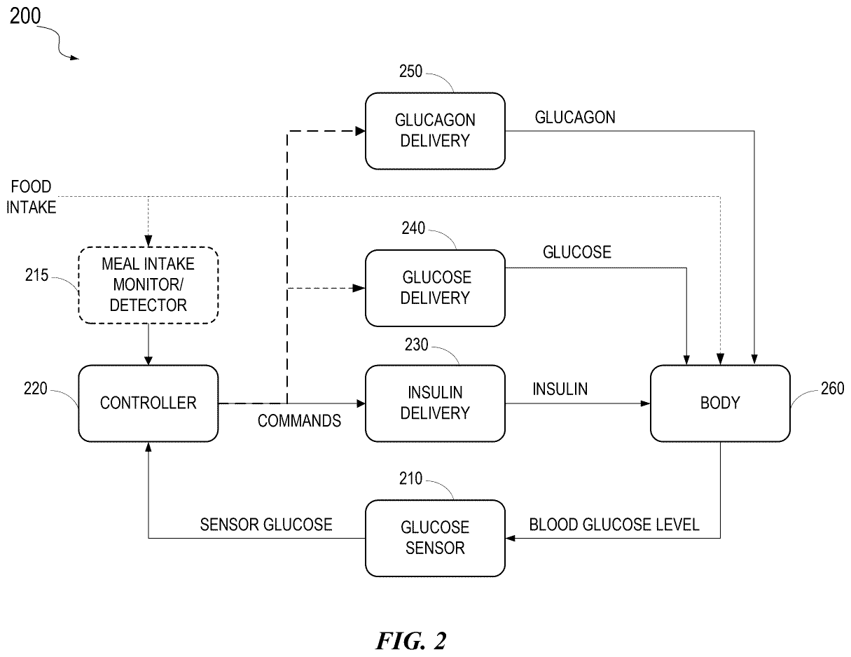

FIG. 2 is a block diagram of an example of a blood glucose level management system 200 according to certain embodiments. In the illustrated example, blood glucose level management system 200 may include a glucose sensor subsystem 210, a controller 220, an insulin delivery subsystem 230, a glucose delivery subsystem 240, and a glucagon delivery subsystem 250. Glucose sensor subsystem 210 may generate sensor glucose (SG) signals (e.g., SG levels) that may be the estimations of blood glucose levels in a body 260, and may provide the SG signals to controller 220. Controller 220 may receive the SG signals and generate commands to insulin delivery subsystem 230, and, in some implementations, glucose delivery subsystem 240 and/or glucagon delivery subsystem 250. Insulin delivery subsystem 230 may receive commands from controller 220 and deliver insulin to body 260 according to the commands. In some embodiments, glucose delivery subsystem 240 may receive commands from controller 220 and provide glucose into body 260 according to the commands. In some embodiments, glucagon delivery subsystem 250 may receive commands from controller 220 and deliver glucagon into body 260 according to the commands.

In some implementations, glucose sensor subsystem 210 may include a glucose sensor, sensor electronics configured to generate SG signals, a sensor communication system configured to send the SG signals to controller 220, and a housing for the sensor electronics and the sensor communication system. The glucose sensor may measure blood glucose levels, for example, directly from a blood stream, or indirectly via interstitial fluid using a subcutaneous sensor as described in more detail below.

Controller 220 may include electrical components and software to generate commands for insulin delivery subsystem 230, glucose delivery subsystem 240, and/or glucagon delivery subsystem 250. Controller 220 may include a controller communication system to receive the sensor signal and provide the commands to insulin delivery subsystem 230, glucose delivery subsystem 240, and/or glucagon delivery subsystem 250. In some implementations, controller 220 may implement a glucose calculator. In some implementations, controller 220 may include a user interface and/or operator interface (not shown) comprising a data input device and/or a data output device. Such a data output device may, for example, generate signals to initiate an alarm and/or include a display or printer for showing status of controller 220 and/or a patient's vital indicators. Such a data input device may include dials, buttons, pointing devices, manual switches, alphanumeric keys, a touch-sensitive display, combinations thereof, and/or the like for receiving user and/or operator inputs. Such a data input device may be used for scheduling and/or initiating insulin bolus injections for meals, for example. It should be understood, however, that these are merely examples of input and output devices that may be a part of an operator and/or user interface and that claimed subject matter is not limited in these respects.

Insulin delivery subsystem 230 may include, for example, an infusion device and/or an infusion tube to infuse insulin into body 260. Similarly, glucose delivery subsystem 240 may include, for example, an infusion device and/or an infusion tube to infuse glucose into body 260. Likewise, glucagon delivery subsystem 250 may include, for example, an infusion device and/or an infusion tube to infuse glucagon into body 260. In some embodiments, the insulin, glucagon, and/or glucose may be infused into body 260 using a shared delivery system and/or infusion tube. In some embodiments, the insulin, glucagon, and/or glucose may be infused using an intravenous system for providing fluids to a patient (e.g., in a hospital or other medical environment). It should be understood, however, that certain example embodiments may include an insulin delivery subsystem 230 without a glucagon delivery subsystem 250 and/or without a glucose delivery subsystem 240. In some embodiments, each of insulin delivery subsystem 230, glucose delivery subsystem 240, and glucagon delivery subsystem 250 may include infusion electrical components to activate an infusion motor according to the commands from controller 220, an infusion communication system to receive commands from controller 220, and a delivery subsystem housing.

In some embodiments, controller 220 may be housed in a delivery subsystem housing, and an infusion communication system may comprise an electrical trace or a wire that carries the commands from controller 220 to the delivery subsystem. In some embodiments, controller 220 may be housed in a sensor system housing, and a sensor communication system may comprise an electrical trace or a wire that carries the sensor signal from sensor electrical components to controller electrical components. In some embodiments, controller 220 may have its own housing or may be included in a supplemental device. In some embodiments, controller 220 may be co-located with a delivery subsystem and a sensor system within a single housing. In some embodiments, a sensor, a controller, and/or infusion communication systems may utilize a cable; a wire; a fiber optic line; RF, IR, or ultrasonic transmitters and receivers; combinations thereof; and/or the like instead of electrical traces, just to name a few examples.

In some embodiments, blood glucose level management system 200 may also include a meal intake monitoring subsystem 215. Meal intake monitoring subsystem 215 may be used to log the amount of user food intake, or may automatically detect the amount of user food intake. For example, in some implementations, the user may enter the food items and/or the estimated amount of carbohydrates in a meal at the meal time. In some implementations, meal intake monitoring subsystem 215 may include sensors (e.g., cameras or accelerators) that may automatically detect a meal event, the food items in a meal, and/or the estimated amount of carbohydrates in the meal. The estimated amount of carbohydrates in the meal may be sent to controller 220, which may determine an appropriate amount of meal bolus and generate a command for insulin delivery subsystem 230 to deliver the meal bolus. In some implementations, meal intake monitoring subsystem 215 may not be used in blood glucose level management system 200, and the dosage for the meal bolus may be determined based on the measured glucose level.

In order to provide users with better user experience, better support, and better therapeutical results, various data associated with the use of the medical devices may be collected before, during, and/or after the use of the medical devices, and the collected data may be used by a medical device data management system to provide insights, training, customer support, troubleshooting, coaching, guidance, and the like, to the user. For example, data related to the user onboarding process, the initial setup of a medical device, user feedback, user interactions with the support or customer service team, user interactions with health care providers, user activities, user meal intakes, user glucose levels, insulin delivery information (e.g., basal and bolus insulin), therapeutical results, alerts or notifications, and the like, may be collected and saved to a data repository. The collected data may then be used to generate insights or other meaningful information related to the use of the medical device, such as user experience scores, leading user experience indicators, patient categories, insight prioritization, and the like. In some embodiments, the insights may be fed into a large language model (LLM) that is trained to, for example, generate personalized recommendations for proactive interventions to improve user experience, suggest precision troubleshooting procedures, create customized educational content and training program, provide motivational messages and coaching instructions, and the like. The medical device data management system may provide a centralized interface for support teams to view insights and recommendations, may automatically deliver personalized insights and interventions through emails, text messages, web apps, chatbots, or other user-preferred communication channel, and/or may provide insights and recommendations to health care providers through emails, text messages, web apps, chatbots, and the like.

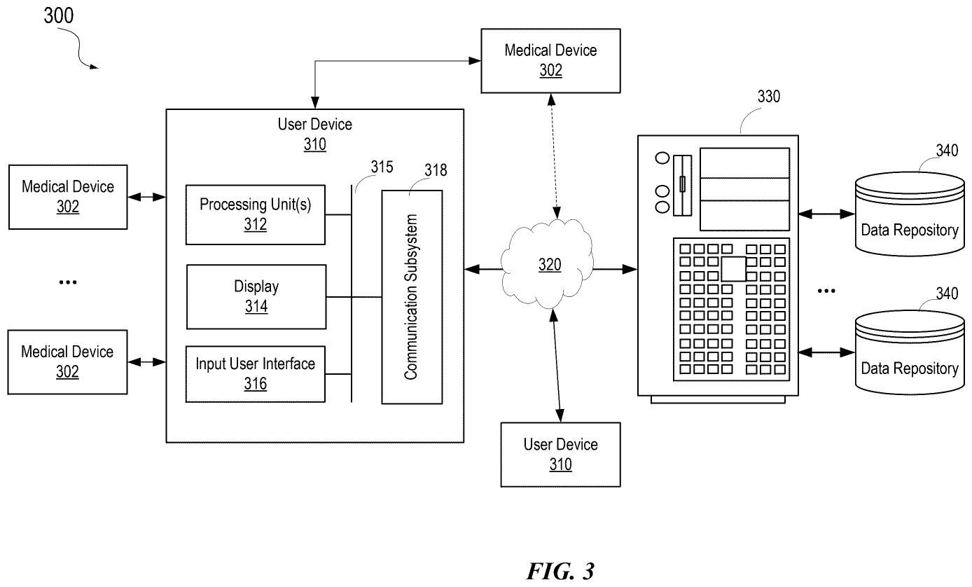

FIG. 3 includes a block diagram illustrating an example of a medical device data management system 300 according to certain embodiments. Medical device data management system 300 may include one or more servers 330 communicatively connected to one or more user devices 310 and/or one or more medical devices 302 through a network 320. The one or more servers 330 may host or may be connected to one or more data repositories 340, which may store collected data, processed data, generated insights, and the like. The one or more servers 330 may process the collected data by, for example, applying the collected data to one or more machine learning models implemented on the one or more servers 330. In some examples, the one or more servers 330 may host one or more websites and/or one or more web-based (or cloud-based) applications. The one or more servers 330 may be accessed by, for example, patients, caregivers, patient parents, healthcare providers, medical device provider personnels (e.g., technical support or customer service team), and the like. In one example, the one or more servers 330 may be owned, operated, or rented (e.g., from a cloud service provider) by a medical device manufacturer or medical device distributor.

Network 320 may include a combination of one or more communication networks, such as the Internet, a Wide Area Network (WAN), a local area network (LAN) (e.g., IEEE 802.11x network), a personal area network (PAN) (e.g., Bluetooth network, an IEEE 802.15x network, etc.), a WiMAX (IEEE 802.16) network, a cellular network, an optical network, a cloud computing network, and the like, or a combination thereof. Network 320 may enable wired and/or wireless communications among the devices and servers.

User devices 310 may include, for example, a user terminal, a personal computer, a laptop, a mobile device (e.g., a smartphone or a touch pad), a smart watch, a client system, a server, a medical device, or another computing device. For example, user device 310 may be a computing device that includes one or more processing units 312, a display 314 (or another output user interface), and an input user interface 316 (e.g., a keyboard, buttons, touch sensor, camera, fingerprint reader, barcode scanner, etc.). User device 310 may also include a communication subsystem 318 that may be used to access network 320 wirelessly or through a wire (e.g., a cable) to communicate with other devices and servers in the system through network 320, and/or may be used to communicate with other devices (e.g., medical devices 302) directly. Processing units 312, display 314, input user interface 316, and communication subsystem 318 may be connected through one or more buses 315. In some examples, user devices 310 may include sensors such as motion sensors, gesture sensors, accelerometers, cameras, and the like. The sensors may be used to detect user activities, such as meal events, exercise, sleep, and the like. In some examples, the user devices may include other sensors such as temperature sensors, blood pressure sensors, blood oxygen sensors, heart rate sensors, respiration rate sensors, electrocardiogram (ECG) sensors, and the like, and may be capable of measuring physiological conditions or vital signs of users.

In the illustrated example, at least some user devices 310 may be communicatively coupled to one or more medical devices 302, such as CGM sensors, other analyte sensors, insulin pumps, insulin pens, or other fluid or medication delivery devices. In some examples, medical devices 302 may include blood pressure sensors, blood oxygen sensors, heart rate sensors, respiration rate sensors, electrocardiogram (ECG) sensors, and the like. A medical device 302 may communicate with a user device 310, a server 330, or another medical device 302. For example, a medical device may communicate with a server 330 through network 320, or may communicate with a server 330 through a user device 310 (or another medical device) and network 320. In one example, a medical device may be a CGM sensor that may communicate with a user device 310 directly or may communicate with a user device 310 through another medical device 302, such as an insulin pump. In some examples, a user device 310 may be used to control a medical device (e.g., a medication delivery device), or process and display data measured by a medical device (e.g., glucose levels of a user). In some examples, a medical device 302 may directly communicate with a user device 310 through, for example, a Bluetooth communication link. In some examples, a medical device 302 may communicate with a user device through, for example, a local area network, such as a Wi-Fi network.

User devices 310 and medical devices 302 may collect various data associated with user interactions with user devices 310 and/or medical devices 302, operations and conditions of medical devices 302, physiological conditions of users measured by user devices 310 and/or medical devices 302, user activities, and the like. The data may be sent to one or more servers 330, which may store the data in one or more data repositories 340 for later processing, or may process some collected data in real time. In some examples, the one or more servers 330 may implement one or more machine learning models (e.g., ensemble models) to process the collected data. Results of the data processing by the one or more servers 330 may be stored in one or more data repositories 340, and may be accessible by users through user devices 310, network 320, and one or more servers 330. In some examples, the one or more servers 330 may, based on the results of the data processing, provide real-time alerts or other messages or information to users through network 320 and user devices 310 using, for example, emails, text messages, web app, chatbot, social media, and the like.

In some examples, the one or more servers 330 may implement or execute one or more large language models that utilize the results of the data processing to provide support, guidance, instructions, recommendation, training, insights, and the like, to patients, caregivers, or healthcare providers. For example, the one or more servers 330 may generate personalized recommendations for proactive interventions to improve user experience, suggest precision troubleshooting procedures, create customized educational content and training program, provide motivational messages and coaching instructions, and the like.

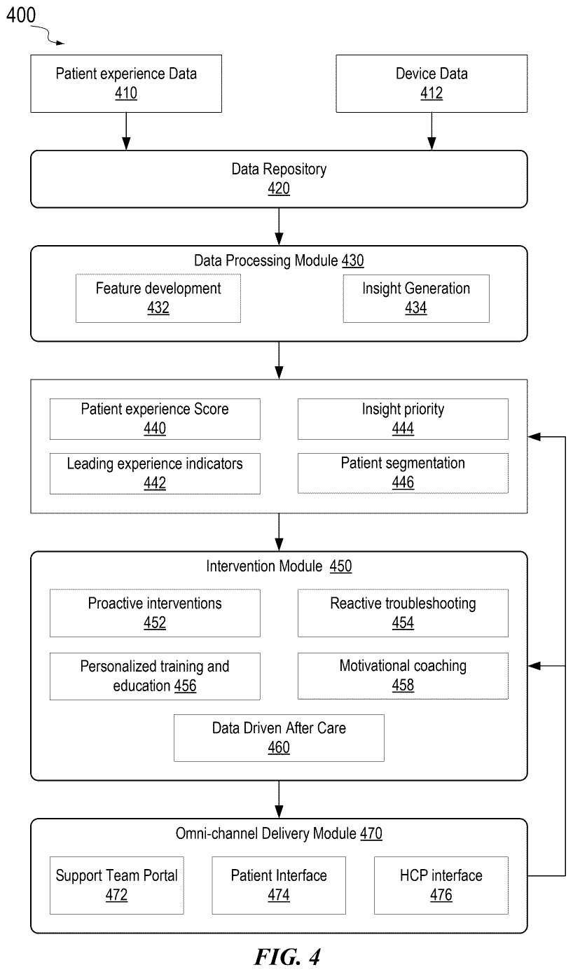

FIG. 4 illustrates an example of a process 400 of medical device data management according to certain embodiments. FIG. 4 is for illustration purposes only, where process 400 may be performed by, for example, one or more servers 330 in combination with one or more data repositories 340. In the illustrated example, data associated with medical devices and users of medical devices may be collected and stored in at least one data repository 420, which may be hosted on a server or on a cloud.

The data associated with the medical devices and the users may include, for example, patient experience data 410 and device data 412. Patient experience data 410 may include, for example, data of patient interactions with medical device, patient feedback data, support tickets, and other data of the users. The patient feedback data may include, for example, user survey data, user complaints, data regarding new users, long-time users, and users that switched from or to other medical devices on the market, and the like. In some examples, patient experience data 410 may include patient demographics (e.g., age, gender, race, etc.) or other characteristics of the users. In some examples, patient experience data 410 may also include data related to the ordering, shipment, and/or warranty of the medical devices. In some examples, patient experience data 410 may also include user training data, such as data regarding the specific training sessions that a user has completed.

Device data 412 may include data from medical devices, such as glucose sensors, insulin pumps, and the like. In some examples, the data from the medical devices may include user physiological condition data, such as data related to user glucose level, body temperature, ECG, blood pressure, blood oxygen level, heart rate, respiration rate, and the like. For example, the data from the medical devices may include CGM data collected over a period of time, and certain metrics of the CGM data, such as the percentage of time during which the user's glucose level is within a target range, the percentage of time during which the user's glucose level is higher than an upper threshold, the percentage of time during which the user's glucose level is below a lower threshold, and the like. In some examples, the data from the medical devices may include medication delivery data, such as bolus insulin time and dose, basal insulin rate, and the like. In some examples, the data from the medical devices may include operating conditions, and alerts, alarms, or other messages provided by the medical devices to users. For example, the data from the medical devices may include the sensitivity of a glucose sensor over a period of time, the accuracy of an analyte sensor over a period of time, the battery level of a medical device over time, and the like. Examples of patient experience data 410 and device data 412 described above are for illustration purposes only. Patient experience data 410 and device data 412 may include any other data associated with users and medical devices.

Data repository 420 may implement one or more databases that store patient experience data 410 and device data 412 according to certain data structures, such that the data can be more efficiently queried and analyzed. For example, the data may be stored according to a hierarchical structure, stored according to data types or categories, stored according to user categories, stored according to time periods, or a combination thereof.

A data processing engine 430 may be implemented using one or more servers or other computing devices to process the collected data to derive insights and other information from the collected data. The insights may include, for example, estimated patient experience scores, leading factors (e.g., features or parameters) that may affect patient experience, the priority of the leading factors, patient segmentation or categories, and the like. In some examples, data processing engine 430 may implement one or more machine learning models to process the data. In the illustrated example, data processing engine 430 may include a feature development engine 432 and an insight generation engine 434.

Feature development engine 432 may process raw data to develop or extract meaningful features that may be used by insight generation engine 434. The processing performed by feature development engine 432 may include, for example, data aggregating, data normalizing, data filtering, data imputation, data reformatting, data embedding, feature extraction, feature deriving, feature combining or aggregating, data encoding, transforming data into formats suitable for machine learning models, and the like. In some examples, feature development engine 432 may filter collected data, format the data for feeding to a machine learning model, embed data samples, extracting certain features from the data samples, and the like. In some examples, feature development engine 432 may remove or mitigate certain artifacts of the data samples, or normalize the data so that the data may be based on the same scale. Outputs of feature development engine 432 may be stored in data repository 420 for later use by insight generation engine 434, or may be directly fed to insight generation engine 434 for further processing. In some examples, feature development engine 432 may be optional, or may be combined with insight generation engine 434.

Insight generation engine 434 may extract insights or other useful information from data stored in data repository and/or outputs of feature development engine 432. Insight generation engine 434 may implement one or more machine learning models, which may include one or more ensemble models. For example, insight generation engine 434 may include one or more ML models for estimating patient experience scores associated with the use of medical devices by users, and one or more ML models or other techniques for determining leading factors that may affect patient experience score and prioritizing the factors for personalized patient interventions to improve patient experience score. The leading factors may be personalized leading factors for individual patients or may be leading factors for a population, such as a group of patients having one or more common characteristics. In some examples, insight generation engine 434 may implement one or more classification models or classification techniques to classify or segment patients based on certain criteria, such as the patient experience scores and leading factors for different patients. In some examples, an eXtreme Gradient Boosting (XGBoost) model, a survival analysis technique, a hidden Markov model (HMM), or another model may be used to process the extracted, derived, and/or aggregated features to predict patient experience score. In some examples, SHapley Additive explanation (SHAP) techniques, Local Interpretable Model-agnostic Explanations (LIME) technique, and the like may be used to identify leading factors that may contribute to the patient experience score, and/or determine personalized user interventions, as described in detail below.

Outputs of data processing engine 430 may include, for example, patient experience score 440, leading experience indicators 442, insight priority information 444, patient segmentation information 446, and the like. Patient experience score 440 may indicate the degree of success and satisfaction of a patient using a medical device, and may be a quantitative value (e.g., a number between 0 and 100 or between 0 and 10), a qualitative value (e.g., low, medium, or high), a vector, another metric, or a combination of multiple metrics.

Leading experience indicators 442 may be determined based on, for example, SHAP values or other feature importance metrics associated with different features or factors. Leading experience indicators 442 may indicate leading factors that may affect user experience, such as the purchasing experience, ease of use, accuracy of measurement results, therapeutical results, the percentage of time during which the patient's glucose level is within a target range, life time of the medical device, frequency of error or malfunction of a medical device, repeated issues, and the like. For example, a patient may have reported the sensor disconnection issue multiple times and may have not reported or complained about other negative experience, and thus the sensor disconnection issue may be one leading experience indicator for this patient. Another patient may have complained about the delivery time of the medical device after placing an order, which may be one leading experience indicator for this patient. The leading experience indicators may include factors that may have positive impact on a patient's experience score, factors that may have negative impact on a patient's experience score. Some features or factors of the collected data may have no or minimum impact on a patient's experience score. As described above, the leading experience indicators may be patient specific, and thus may be different from patient to patient, due to different characteristics of the patients (e.g., age, life style, medical condition, length of using a type of medical device, etc.).

In addition to identifying leading experience indicators for a patient, data processing engine 430 (e.g., insight generation engine 434) may also rank or prioritize the insights or leading experience indicators 442 based on, for example, the potential impact of the leading experience indicators on the patient experience score, where the potential impact may be determined based on, for example, SHAP values or other feature importance metrics. Insight priority information may be used to more quickly and efficiently improve patient experience score, for example, by performing user interventions associated with the insights or experience indicators that have higher priority and have more significant impact on the patient experience score.

In some examples, data processing engine 430 (e.g., insight generation engine 434) may also classify or segment patients based on certain criteria, such as patient demographic information, patient life style, patient health condition, patient experience scores, leading experience indicators of the patient, and the like. Patient segmentation information 446 may be used for targeted user interventions to more efficiently improve patient experience scores of individual patients. In some examples, the patient experience scores of patients in a same segment (or class or category) may be improved through similar user interventions, and thus interventions that have improved the patient experience score for a first patient may be provided to a second patient in the same segment as the first patient.

Intervention engine 450 may use the insights generated by data processing engine 430 to determine appropriate patient interventions, either proactively or reactively, for example, based on issues identified as causes of a low patient experience score of a patient. In one example, intervention engine 450 may use one or more large language models to determine appropriate patient interventions. The insights generated by data processing engine 430 and/or data or developed features stored in data repository 420 may be used as inputs or prompts to a large language model. For example, the generated insights may be encoded into prompts to a large language model, and the large language model may process the encoded insights to generate intervention candidates that can be manually selected or automatically filtered according to certain criteria, such as some predefined medical safety rules (e.g., maximum medication dosage, minimum glucose level, etc.). For example, the encoding may include features associated with the top SHAP values and raw feature values, and the large language model (or a classification model) may provide a classification prediction to match the patient with the appropriate intervention. The large language model may suggest proactive interventions 452 by generating personalized recommendations for improving patient experience. The personalized recommendations may include, for example, recommended adjustments to device settings, proactive health tips, early warnings about potential issues, and the like. The large language model may also support reactive troubleshooting 454 by suggesting precision troubleshooting steps based on the specific issues identified by insight generations engine 434 (e.g., based on the SHAP values). The large language model may also provide personalized training and education content 456 by, for example, creating customized educational content and training programs for individual patients. The large language model may also provide motivational coaching information 458, for example, by generating motivational messages and coaching to encourage patient to adhere to recommended treatment plans and suggested lifestyle changes. In some examples, the large language model may also provide data-driven aftercare 460, for example, by personalizing follow-up care for a patient based on ongoing data analysis.

The filtered intervention candidates may be provided to various users through one or more communication channels or user interfaces. In the example shown in FIG. 4, omni-channel delivery engine 470 may provide a unified platform for personalized interventions. For example, omni-channel delivery engine 470 may provide a support team portal 472, a patient interface 474, a health care provider (HCP) interface 476, and the like, to provide various information (e.g., interventions, insights, instructions, recommendations, etc.) to various entities through various communication channels. As described above, in some examples, omni-channel delivery engine 470 may actively deliver the information to the various entities through, for example, emails, text messages, social media, chatbots, user applications, or other user-preferred communication channels. In some examples, omni-channel delivery engine 470 may provide the information to users on demand, by allowing user to access web-based applications, cloud-based applications, and/or websites. For example, support team portal 472 may provide a centralized interface for support teams to view insights and recommendations for a patient, thereby customizing the support and other interactions with the patient. Patient interface 474 may deliver personalized insights and interventions to a patient through email, text, web app, chatbot, or other preferred communication channels. HCP Interface 476 may provide insights and recommendations to health care providers of a patient through email, text, web app, chatbot, or other preferred communication channels.

In some examples, the generated insight, performed interventions, and/or results of the interventions (e.g., patient outcome) may be used as new data points for improving the various models used in process 400. For example, the new data points may be used to calibrate some model outputs, or retrain or tune some model parameters. In some examples, the prompt encoding and/or prompt generation using large language models may be updated using labels derived from the patient outcomes. In one example, an initial prompt may be used to select, from pre-defined interventions, one or more pre-defined interventions for each person, and the interventions may be fine-tuned over time based on downstream results. The fine-tuning can be done at the prompt level (e.g., by tweaking the actual interventions) or can be done further upstream by relabeling data based on user feedback and interactions during interventions.

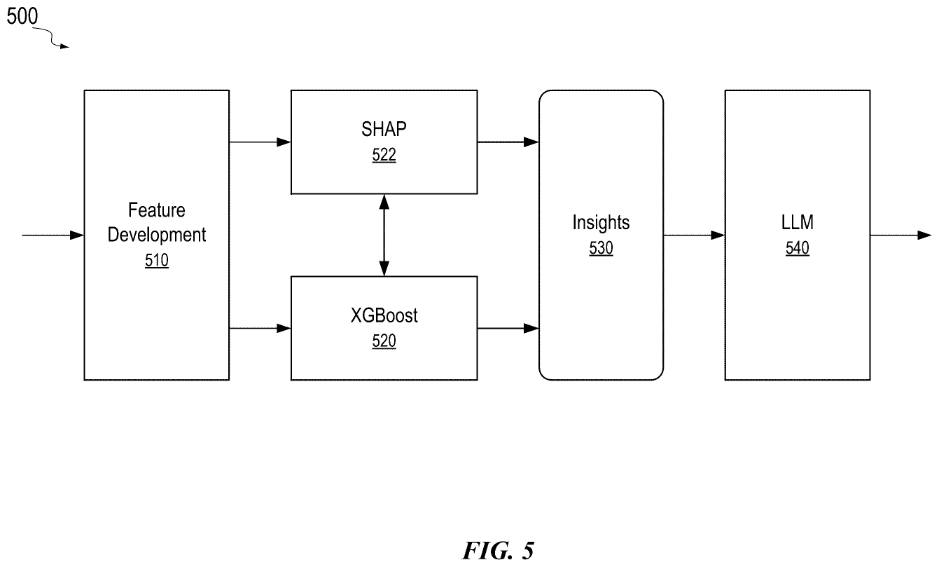

FIG. 5 illustrates a simplified example of an implementation of a medical device data management system 500 disclosed herein according to certain embodiments. Even though not shown in FIG. 5, medical device data management system 500 may include one or more data repositories for storing raw data and/or processed data. In the illustrated example, medical device data management system 500 may include a feature development engine 510, which may be similar to feature development engine 432 and may pre-process collected user data and device data so that the data may be suitable for input into machine learning models for insights generation, leading experience factor determination, and the like. As described above, feature development engine 510 may perform, for example, data aggregating, data normalizing, data filtering, data selection, data reformatting, data embedding, feature extraction, data transformation, and the like, such that the pre-processed data can be used by the machine learning models for insight generation. In some examples, feature development engine 510 may filter collected data, format the data for feeding to an ML model, embed data samples, extract certain features from the data samples, and the like. In one example, feature development engine 510 may select data samples, remove or mitigate certain artifacts of the data samples, extract certain features of the data samples, assemble the data samples according to a data structure of the inputs to a machine learning model, and the like.

In the illustrated example, medical device data management system 500 may include one or more XGBoost engines 520 for insight generation and one or more SHAP engines 522 for determining SHAP values associated with input parameters and/or features extracted by feature development engine 510. In some implementations, XGBoost engines 520 and SHAP engines 522 may be implemented using a same program or a same piece of code, or may be implemented using different code and may be executed together in parallel or sequentially. An XGBoost engine 520 may implement an XGBoost model. In one example, an XGBoost engine may be used to predict the patient experience score. The XGBoost model may be trained on historical data that may include patient outcomes and satisfaction scores and associated user data and device data samples. The trained XGBoost model may then be used to estimate a patient experience score based on new user data and device data samples.

XGBoost is a scalable, distributed gradient-boosted decision tree (GBDT) machine learning technique. The XGBoost technique may build a predictive model by combining the predictions of multiple individual models, such as decision trees, in an iterative manner. The technique may sequentially add weak models to an ensemble, with each new model focusing on correcting the errors made by the existing ones. The XGBoost technique uses a gradient descent optimization technique to minimize a predefined loss function during training. The XGBoost technique can handle complex relationships in data, prevent overfitting, and incorporation of parallel processing for efficient computation. The XGBoost technique provides parallel tree boosting and may be used, for example, for regression, classification, and ranking tasks, where the decision trees of an XGBoost model may be used for classification to predict a category, may be used for regression to predict a continuous numeric value, or may be used for comparison to determine ranking or priority. For example, decision trees may be used herein to generate insights by evaluating a tree of true/false feature questions connected by if-then-else relations, and estimating the minimum number of questions needed to assess the probability of making a correct decision. Thus, an XGBoost model may be an ensemble machine learning model that may combine many models and use gradient boosting techniques for decision trees.

Boosting techniques may be different from other ensemble learning techniques as the boosting techniques may build a sequence of initially weak models into increasingly more powerful models. In boosting, the trees may be built sequentially such that each subsequent tree may reduce the errors of the previous tree. Each tree learns from its predecessors and updates the residual errors. Hence, the tree that grows next in the sequence may learn from the residual errors. The base models in boosting can be weak models in which the bias may be high, and the predictive power of the base models may only be slightly better than random guessing. Each of these weak models may contribute some vital information for prediction, enabling the boosting technique to produce a strong learner by effectively combining these weak models. The final strong model may have both a low bias and a low variance. In contrast to bagging techniques such as random forest where trees are grown to their maximum extent, boosting may use trees with fewer splits. The small trees, which may not be very deep, can be highly interpretable. Parameters such as the number of trees or iterations, the rate at which the boosting learns, and the depth of the tree, may be selected through validation techniques such as k-fold cross validation.

Gradient boosting is an extension of boosting, where the process of additively generating weak models is formalized as a gradient descent algorithm over an objective function. Gradient boosting techniques may choose how to build a more powerful model using the gradient of a loss function (e.g., residual errors) that determines the performance of a model. The gradient boosting techniques boost or otherwise improve a weak model by combining it with a number of other weak models in order to generate a collectively strong model. For example, gradient boosting may set targeted outcomes for the next model in an effort to minimize errors. The targeted outcomes may be set based on the gradient of the errors. The gradient-boosted decision tree (GBDT) machine learning techniques may iteratively train an ensemble of shallow decision trees, with each iteration using the error residuals of the previous model to fit the next model. The final prediction can be, for example, a weighted sum of all of the tree predictions.

In one example of the gradient boosting ensemble technique, an initial model may be defined to predict a target variable. The initial model may be associated with a residual. A new model may be fit to the residual. The initial model and the new model may be combined to form a boosted model of the initial model. The mean squared error from the boosted model may be lower than that from the initial model. To further improve the performance of the boosted model, the residual of the boosted model may be determined and fitted to create a new boosted model. This process can be performed for many iterations, until the residual is minimized to a value lower than a target value. Thus, the additive learning process may not disturb the models created in the previous steps, and may bring down the error or residual by adding new models.

The extreme gradient boosting (XGBoost) technique is a scalable and highly accurate implementation of gradient boosting. XGBoost may utilize decision trees as base models and employ regularization techniques to enhance model generalization. In XGBoost techniques, trees can be built in parallel, instead of sequentially as in other GBDT techniques. XGBoost may be different from other gradient boosting techniques as it may use a second-order approximation of a scoring function. The XGBoost techniques may use a level-wise strategy, scanning across gradient values and using partial sums to evaluate the quality of splits at every possible split in the training set. An XGBoost model may examine the input under various “if” statements (vertices in the tree graph). Whether the “if” condition is satisfied influences the next “if” condition and the eventual prediction. The XGBoost technique may progressively add more and more “if” conditions to the decision tree to build a stronger model.

The one or more SHAP engines 522 for determining SHAP values associated with input parameters and/or features extracted by feature development engine 510 may be used to explain individual predictions by calculating the contribution of each feature. SHAP values indicate how each feature affects each final prediction, the significance of each feature compared to others, and the model's reliance on the interaction between features. The SHAP values are from coalitional game theory. A SHAP value may be the average marginal contribution of a feature across all possible combinations or coalitions. A large positive SHAP value for a feature may indicate that the feature affects the model's prediction more than the average model prediction. A prediction can be explained by, for example, assuming that each feature is a player in a game where the prediction is the payout, and the SHAP values indicate how to fairly distribute the payout among the features. SHAP values may be calculated for each prediction made by the XGBoost model to provide insights into which features (e.g., device readings, interaction data, etc.) may have an impact on the patient experience score. Thus, patient experience scores may be determined based on the prediction of the XGBoost model, while the SHAP values may help in interpreting these scores by highlighting the most influential factors.

The SHAP technique is a local explanation method that gives an explanation for why a model made a specific prediction, rather than trying to explain the overall or general behavior of a model. SHAP is a model-agnostic explanation method that may be used to explain the predictions of any machine learning model that takes inputs and predicts outputs, including linear regression, decision trees, random forests, gradient boosting models, neural networks, and the like. The goal of SHAP is to explain the prediction of an instance x by computing the contribution of each feature to the prediction. The SHAP explanation method computes the Shapley values that indicate how to fairly distribute a prediction (“payout”) among the features (“players”). A player can be an individual feature, or a group of features. For example, to explain an image, pixels can be grouped to macro-pixels and the prediction may be distributed among the pixels or macro-pixels. The SHAP technique may specify the explanation as:

$$g ( z ) = {ϕ}_{0} + {\sum }_{j = 1}^{M} {ϕ}_{j} {z}_{j} ,$$

where g is the explanation model, z∈{0,1}^{M} is the coalition vector, M is the maximum coalition size, and ø, is the feature attribution (e.g., the SHAP value) for a feature j.

The outputs of the one or more XGBoost engines 520 and the one or more SHAP engines 522 may be insights 530, which may include features that may affect the patient experience score, SHAP values or other feature importance metrics of the features, leading indicators of patient experience score determined based on the SHAP values, priority of the insights determined based on their potential impact on patient experience as indicated by SHAP values, and the like. Insights 530 may be stored in a data repository for use by an LLM 540 for patient intervention and omni-channel information delivery to various people, such as patients, caregivers, healthcare providers, and medical device company personnel (e.g., sales, customer service, and/or technical support team members).

LLM 540 may be a computational model capable of language generation or other natural language processing tasks. LLM 540 may include a generative artificial intelligence (AI) model. In one example, LLM 540 may be based on a transformer architecture (e.g., including one or more transformer models), and may be trained using, for example, reinforcement learning from human feedback (RLHF). Examples of LLM 540 may include, for example, OpenAI GPT-1, 2, 3, 4, 4o, Meta Large Language Model Meta AI (LLaMa), Google Gemini, Anthropic Claude, Mistral AI Mixtral, and the like. LLM 540 may, based on certain inputs and/or prompts, perform text generation functions, such as drafting emails, providing training or education, posting on blogs, chatting with users, providing step-by-step instructions or guidance, trouble-shooting a malfunctioned medical device, and the like. LLM 540 may also analyze text to determine the customer's tone, mode, or sentiment, in order to correspond with the user in an appropriate manner to improve user experience. LLM 540 may have translation or multilingual capability and can provide personalized interventions to users across the world using user preferred languages.

For example, as described above, LLM 540 may suggest proactive interventions by generating personalized recommendations for improving patient experience. The personalized recommendations may include, for example, adjustments to device settings, proactive health tips, early warnings about potential issues, and the like. LLM 540 may also support reactive troubleshooting by suggesting precision troubleshooting steps based on the specific issues identified by the one or more XGBoost engines 520 and the one or more SHAP engines 522. LLM 540 may also provide personalized training and education by, for example, creating customized educational content and training programs for individual patients. LLM 540 may also provide motivational coaching, for example, by generating motivational messages and coaching to encourage patient to adhere to recommended treatment plans and suggested life style changes. In some examples, LLM 540 may also provide data-driven aftercare, for example, by personalizing follow-up care for a patient based on ongoing data analysis.

The omni-channel delivery may include actively delivering the information to various entities through, for example, emails, text messages, social media, chatbots, user applications, and other communication channels. In some examples, the omni-channel delivery platform may provide the information to users on demand by allowing user to access web-based applications, cloud-based applications, and/or websites. For example, the omni-channel delivery platform may provide a centralized interface for support teams to view insights and recommendations for a specific patient, thereby customizing the support and other interactions with the patient. The omni-channel delivery platform may deliver personalized insights and interventions to a patient through email, text, web app, chatbot, or other patient preferred communication channels. The omni-channel delivery platform may also provide data-driven insights and recommendations to health care providers of a patient through email, text, web app, chatbot, or other preferred communication channels. In one example, patients or patient representatives (such as health care providers or caregivers) can access patient data and insights through an online application, a website, or a web-based portal. In certain embodiments, the online application or website may also support user feedback and inputs. For example, the application may allow patients or caregivers to confirm or prioritize the generated insights. Alternatively or additionally, the system can obtain feedback information from other data sources. The feedback can be used to adjust the insights and patient priority accordingly.

The artificial intelligence and machine learning techniques describe above can be used to extract knowledge and insights from data in various forms in order to understand and analyze phenomena represented by the data. The machine learning models may be trained using historical patient data, device data, and patient experience scores. For example, a neural network or other machine learning models can be trained using one or more datasets to extract useful features and insights from similar input data or to make predictions of patient experience scores based on the input data. In some examples, the datasets used for the training may include training samples used to train a machine learning model, and testing samples used to evaluate the model's performance after the training. In general, the quality or performance (e.g., accuracy and sensitivity) of the machine learning model depends heavily on the quantity and quality of the datasets used for the training. Thus, one way to improve the performance of a machine learning model is to improve the quantity and/or quality of the training datasets. In some cases, a large amount of data may be needed to train a model to achieve a sufficiently accurate and robust model.

A machine learning model such as a neural network model, a decision tree, or a transformer may learn parameters of the model (e.g., weights of a neural network) on its own during a training (i.e., learning) process based on some user specified parameters (which may be referred to as hyperparameters), such as the number of filters, the filter size, the architecture of the network, and the like. For example, a neural network may be trained using the back propagation method and appropriate training data. An XGBoost model may be trained using the gradient boosting techniques. The learning may be supervised learning, unsupervised learning, or reinforcement learning. In supervised learning, the training samples may need to be labeled with ground truth or known results (e.g., target classes), such that the model may be tuned to make inferences that match the ground truth or known results of the training samples. For example, an XGBoost model may be trained using supervised learning.

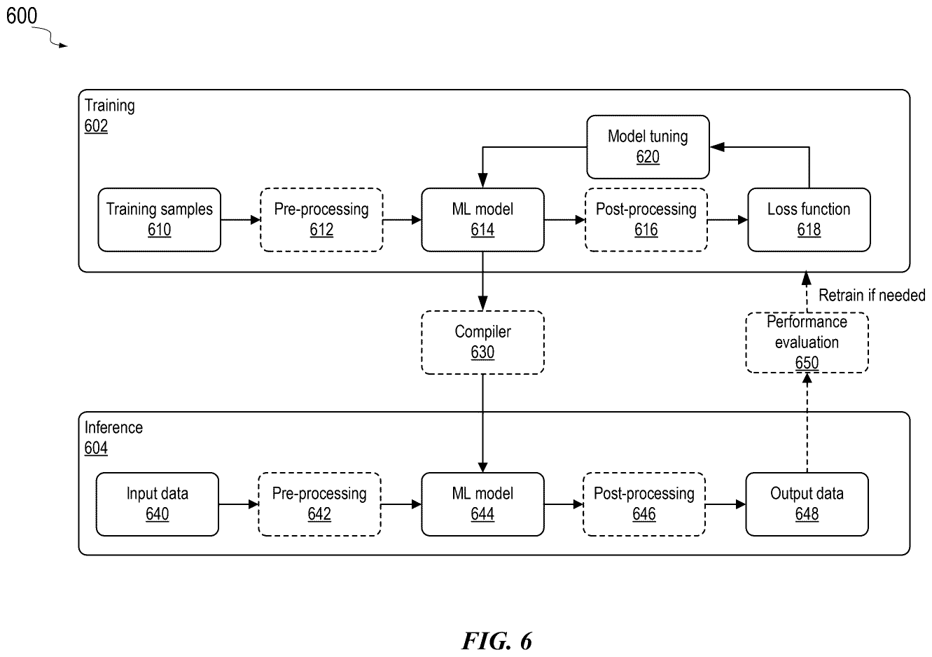

FIG. 6 illustrates an example of a machine learning process 600 that may be used to train the machine learning models used in the medical device data management system disclosed herein according to certain embodiments. Machine learning process 600 may include a training (or learning) phase 602 and an inference (or prediction) phase 604. Training phase 602 and inference phase 604 may be performed by the same computing system or different computing systems. For example, training phase 602 may be performed by a computing system that has higher computing power (e.g., GPUs, TPUs, systolic arrays, etc.) and larger storage space, while inference phase 604 may be performed by a light-weight computing system that may have lower computing power and smaller storage space.

In training phase 602, training samples may be obtained at block 610. The training samples may be labeled or unlabeled (e.g., for unsupervised learning or reinforcement learning). The training samples may include samples for training an ML model and samples for testing the trained ML model. The training samples may optionally be pre-processed at block 612. The pre-processing may include, for example, filtering the training samples, formatting the data of the training samples for feeding to the ML model, embedding the training samples, extracting certain features from the training sample, and the like. In one example, the pre-processing may be performed to remove or mitigate certain artifacts, variability, and the like. The pre-processed training samples may be fed to the ML model, which may be a new model or may be adopted from an existing model (e.g., a pre-trained model).

The ML model may be applied to the training samples to generate outputs at block 614. In some embodiments, the outputs of the ML model may optionally be post-processed at block 616, where the post-processing may include, for example, simplifying, visualizing, majority voting, thresholding, and the like. The results of the ML model or post-processing may be compared with the ground truth or known classifications of the training samples to determine a loss function at block 618. The loss function may indicate the error (loss) between the output of the ML model and the ground truth, and may be used by a training algorithm or optimization algorithm to tune the parameters of ML model at block 620 to reduce the loss. The training/optimization algorithm may include, for example, gradient descent, Newton method, conjugate gradient, quasi-Newton method, Levenberg-Marquardt algorithm, AdamW, NadamW, Heavy Ball, Nesterov, LAMB, Adafactor, distributed Shampoo, and the like.

The ML model may be trained iteratively using the training samples to minimize the loss function for the training samples. The trained ML model with the tuned parameters may be tested using samples that are not used during the training. If the trained ML model can achieve the target performance, the ML model may be deployed to make inferences from real-world data. In some embodiments, the trained ML model may be compiled into executable instructions by a compiler at block 630. The compilation may be based on the available resource of the target computing system for implementing the ML model.

In inference phase 604, new input data to be analyzed may be received at bock 640, and may optionally be pre-processed at block 642 as in training phase 602. The input data may then be fed to the trained ML model at block 644. The output of the ML model may optionally be post-processed at block 646 as in block 616, to generate output data 648. In some implementations, output data 648 may be evaluated at block 650 to determine the performance of the trained ML model. If the trained ML model could not achieve the target performance on new input data, the ML model may be re-trained.

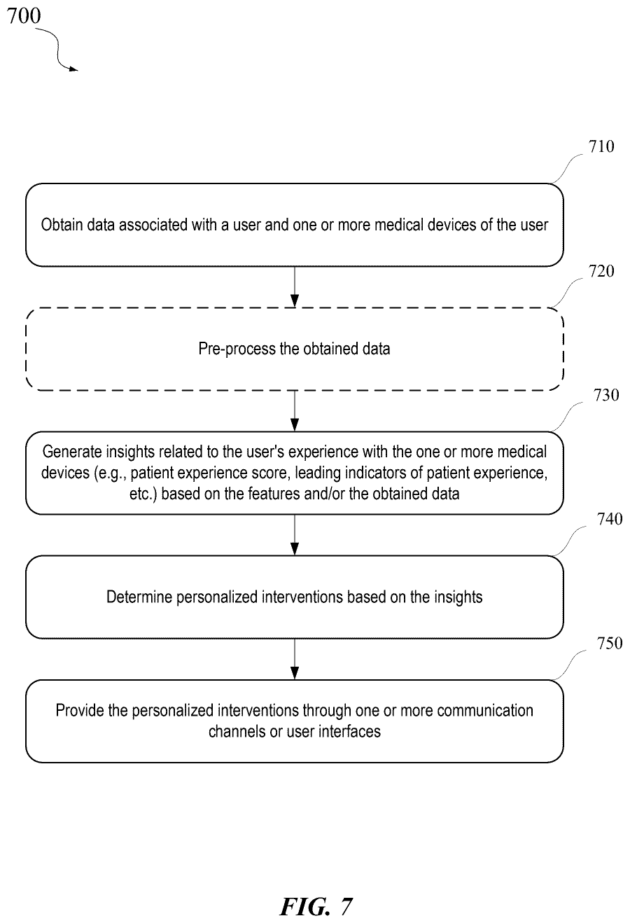

FIG. 7 includes a flowchart 700 illustrating an example of a processor-implemented method for medical device data management according to certain embodiments. Operations in flowchart 700 may be performed using, for example, one or more processors or computing systems (e.g., computing device 106, remote/cloud computing system 108, one or more servers 330, medical device data management system 500, a computing system 800 described below, or a combination thereof), in combination with one or more data storage devices (e.g., one or more data repositories 340), one or more medical devices (e.g., delivery device 102, monitoring device 104, glucose sensor subsystem 210, insulin delivery subsystem 230, or medical devices 302), one or more user devices (e.g., user devices 310), and the like. Although flowchart 700 may describe the operations as a sequential order, some of the operations may be performed in parallel or concurrently. In addition, the order of the operations may be rearranged. The process may have additional steps not included in the flowchart. Some operations may be optional or may be omitted in some implementations. Some operations may be performed more than one time. Some operations may each include multiple sub-operations. Some operations in different blocks may be combined and performed using a same device, processing engine, computer program, machine learning model, or functional module.

In the example illustrated in FIG. 7, the operations in flowchart 700 may include, at block 710, obtaining data associated with a user and one or more medical devices of the user. The data associated with the user and the one or more medical devices of the user may include, for example, health condition data of the user, operation data of the one or more medical devices, the user's feedback associated with purchasing and/or using the one or more medical devices; user interaction data, and the like, or a combination thereof. In one example, the health condition data of the user may include glucose levels of the user and/or other measurement results, such as the body temperature, blood pressure, blood oxygen levels, hear rates, respiration rates, ECG data, and the like. In one example, the operation data of the one or more medical devices may include insulin deliver data of an insulin delivery device, such as basal rates, bolus dosages, bolus time, and the like, and/or may include data such as alerts, warnings, or other messages generated by the one or more medical devices, such as warnings regarding hypoglycemic conditions or hyperglycemic conditions, or warning regarding low battery, low sensitivity, errors, or loss of communication of the one or more medical devices. The user interaction data may include, for example, data associated with user interactions with the one or more medical devices, data associated with user interactions with a provider of the one or more medical devices, or a combination thereof. In some examples, the user interaction data may include data regarding past complaints by the user, support tickets opened for the user, past interactions of the user with the sale, technical support, or customer service personnel, user feedback data, communications between the user and the provider of the one or more medical devices, and the like. The data associated with the user may include, for example, different characteristics of the patients, such as age, life style, medical condition, length of using a type of medical device, preferred communication methods, and the like.

Optional operations in block 720 may include pre-processing the obtained data for feature development. As described above, pre-processing the obtained data may include creating or extracting one or more features from the obtained data, combining or aggregating features, selecting features, filtering the data to remove outliers, imputing missing values, determining values of new parameters based on parameters in the obtained data (e.g., calculate the total daily dose based the basal insulin and bolus insulin delivered), binning, categorizing, or grouping the data, transforming the data (e.g., using mathematical operations to change the distribution or scale of the data), splitting some data, scaling the data (e.g., data normalization or standardization, such as z-score normalization), encoding the data (e.g., numerical representation of text data), reducing the dimensionality of the data, and the like.

Operations in block 730 may include generating insights related to the user's experience with the one or more medical devices based on the features and/or the obtained data. The insights may include, for example, a patient experience score associated with the one or more medical devices, factors (e.g., features or parameters) that affect the patient experience score, Shapley additive explanations (SHAP) values associated with the factors, priority of the factors, a classification of the user, or a combination thereof. The patient experience score may be a numerical value, a categorical or qualitative value, a range, a combination of multiple metrics, and the like. The priority of the factors may be determined based on, for example, the SHAP values associated with the factors. As described above, a SHAP value of a feature may indicate how much the feature may affect the estimation of the patient experience score. The classification or segmentation of the user may be based on, for example, the patient experience score of the user (e.g., users with similar experience score may be in the same category or group), the leading factors that may affect the user's experience score (e.g., ease of use, accuracy, or therapeutical result), the length of using the medical device (e.g., a new user or an experienced user), the preferences of the user (e.g., preferring email communication over text message, no or minimum number of alerts or alarms, etc.), objectives of the user (e.g., seeking better therapeutical results or seeking more guidance or coaching), and the like.

In some examples, generating the insights may include applying the obtained data and/or developed features to a machine learning model trained using historical data. In one example, the historical data may include the data associated with users and the one more medical devices of the users, and the corresponding known patient experiences scores of the users using the one more medical devices, and thus a supervised learning technique may be used to train the machine learning model using the historical data. In some examples, the machine learning model may include an extreme gradient boosting (XGBoost) model trained to predict a patient experience score based on the obtained data and/or the one or more features extracted from the obtained data. In one example, the XGBoost model may be an ensemble model that includes a plurality of machine learning models, such as decision trees. In some examples, generating the insights may include determining Shapley additive explanations (SHAP) values of one or more parameters of the obtained data for predicting a patient experience score. The SHAP values may explain individual predictions by calculating the contribution of each feature to a prediction. In some implementations, XGBoost models in an open-source library (e.g., GitHub) or a proprietary library may be adopted to generating insights related to the user's experience with the one or more medical devices based on the developed features and/or the obtained data. The XGBoost models can be implemented using C++, Java, Python, R, and Julia, Perl, Scala, and the like. Similarly, the SHAP techniques may also be adopted from an open-source library or a proprietary library.

Operations in block 740 may include determining personalized interventions based on the insights. In some examples, determining the personalized interventions for the user based on the insights may include applying the insights and/or collected data to a large language model (LLM) trained to generate the personalized interventions. The large language model may include, for example, one or more transformer models, and may be trained using, for example, reinforcement learning from human feedback (RLHF). Examples of the large language model may include OpenAI GPT-1, 2, 3, 4, 4o, Meta Large Language Model Meta AI (LLaMa), Google Gemini, Anthropic Claude, Mistral AI Mixtral, and the like. The large language model may, based on the collected data and/or insights generated at block 730, perform text (or audio) generation functions, such as drafting emails, providing training or education, posting on blogs, chatting with users, providing step-by-step instructions or guidance, trouble-shooting a malfunctioned medical device, and the like. In some examples, the personalized interventions may be automatically generated without human involvement. For example, based on some insights generated at block 730, a computing system may determine that user interventions may be needed, such as when a predicted patient experience score is low, when a device error is detected, when the health condition of the user is abnormal, or when the measurement results of a medical device are out of range. The computing system may then apply the LLM to generate personalized intervention for the user. In some examples, the LLM may be applied to the insights and/or collected data when a prompt is received, where the prompt may be from a patient, a healthcare provider, a caregiver, a sales, support, or customer service team member, and the like.

The personalized interventions may include, for example, proactive user interventions based on the insights, reactive user interventions for troubleshooting and customer service, personalized user education or training program, motivational coaching to encourage the user, personalized care (e.g., automatic follow-up care), or any combination thereof. The personalized interventions may be determined based on insights such as user classification or segmentations, user preferences, and the like. For example, for a new user or a user on a new therapy, more training, education, or step-by-step instructions may be provided, and more motivation may be provided. For a user that wants to receive fewer alerts, alarms, or other messages, the alerts, alarms, or other messages to the user may be minimized. For example, only messages that are related to the leading factors for the user's patient experience score may be provided. For a user that is more interested in achieving better results, more messages or guidance for achieving better results may be provided. For users that prefer email communication, the personalized interventions may be more in the form of emails. For users that prefer live communication, chatbots or instant messages may be used more often. In some examples, the LLM may analyze text to determine the customer's tone, mode, or sentiment, in order to correspond with the user in an appropriate manner to improve user experience. The LLM may have translation or multilingual capability and can provide personalized interventions to users across the world using user preferred languages.

Operations in block 750 may include providing the personalized interventions through one or more communication channels or user interfaces. The one or more communication channels or user interfaces may include, for example, one or more web applications, one or more user applications, one or more chatbots, emails, text messages, one or more websites, or a combination thereof. The personalized interventions may be provided to a patient, a healthcare provider, a caregiver, a medical device provider personnel, or a combination thereof, using one or more user portals. For example, different portals may be provided to different types of users of the medical device data management system.

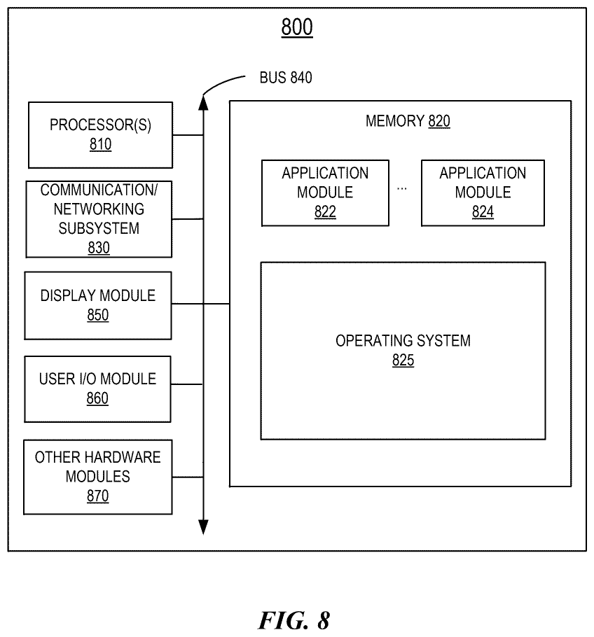

FIG. 8 is a block diagram of an example of an computing system 800 that may implement some of the examples disclosed herein. For example, computing system 800 may be used to implement computing device 106, remote/cloud computing system 108, one or more servers 330, medical device data management system 500, delivery device 102, monitoring device 104, glucose sensor subsystem 210, insulin delivery subsystem 230, medical devices 302, or user devices 310. It should be noted that FIG. 8 is meant only to provide a generalized illustration of various components, any or all of which may be utilized as appropriate. In addition, it can be noted that components illustrated by FIG. 8 can be localized to a single device and/or predict future blood glucose levels. In distributed among various networked devices, which may be disposed at different geographical locations.

In the illustrated example, computing system 800 may include one or more processor(s) 810 and memory 820. Processor(s) 810 may be configured to execute instructions stored in memory 820 for performing one or more of the methods described herein and other applications. Processor(s) 810 may include, for example, one or more central processing units, microprocessors, microcontrollers, special-purpose processors (e.g., digital signal processors), ASICS, DSPs, FPGAs, or other processors suitable for implementation within a portable electronic device. Processor(s) 810 may be communicatively coupled with a plurality of components within computing system 800. To realize this communicative coupling, processor(s) 810 may communicate with the other illustrated components across a bus 840. Bus 840 may be any subsystem adapted to transfer data within computing system 800. Bus 840 may include a plurality of computer buses and additional circuitry to transfer data.

Memory 820 may include one or more transitory and/or non-transitory storage devices, such as, for example, a static random-access memory (SRAM), a dynamic random access memory (DRAM), a read-access memory (RAM), a read-only memory (ROM), a programmable read-only memory (PROM), an erasable programmable read-only memory (EPROM), a FLASH-EPROM, a secure digital (SD) card, and any other memory chip or cartridge. Such storage devices may be configured to implement any appropriate data stores, including without limitation, various file systems, data structures, computer-readable instructions, program modules, and the like. In some embodiments, memory 820 may be distributed into different hardware modules. A set of instructions and/or code might be stored on memory 820. The instructions might take the form of executable code that may be executable by computing system 800, and/or might take the form of source and/or installable code, which, upon compilation and/or installation on computing system 800 (e.g., using any of a variety of generally available compilers, installation programs, compression/decompression utilities, etc.), may take the form of executable code.

Memory 820 may include an operating system 825 loaded therein. Operating system 825 may be operable to initiate the execution of the instructions provided by application modules 822-824 and/or manage other hardware modules 870, as well as interface with a communication subsystem 830 which may include one or more wired and/or wireless transceivers. Operating system 825 may be adapted to perform other operations across the components of computing system 800 including threading, virtualization, resource management, data storage control, and other similar functionality. In some embodiments, memory 820 may store a plurality of application modules 822 through 824, which may include any number of applications. Examples of the applications may include an insulin calculator, a blood glucose level monitor or predictor, a glucose level management application, and the like. Application modules 822-824 may include particular instructions to be executed by processor(s) 810. In some embodiments, certain applications or parts of application modules 822-824 may be executable by other hardware modules 870.

Communication subsystem 830 may include, for example, an infrared communication device, a wireless communication device and/or chipset (such as a Bluetooth® device, an IEEE 802.11 device, a Wi-Fi device, a WiMax device, cellular communication devices, etc.), and/or similar communication interfaces. One or more antennas (not shown) may be used for wireless communication as part of communication subsystem 830 or as a separate component coupled to any portion of computing system 800, such as a wireless charging receiver or a near-field communication receiver. In some embodiments, communication subsystem 830 may include circuits for wired communication technologies, such as Ethernet, coaxial communications, universal serial bus (USB), and the like. In some embodiments, communication subsystem 830 may include transceivers to communicate with base transceiver stations and other wireless devices and access points, which may include communicating with different data networks and/or network types, such as wide-area networks (“WANs”), wireless wide-area networks (WWANs), local area networks (LANs), wireless local area networks (WLANs), personal area networks (PANs), or wireless personal area networks (WPANs). A WWAN may be, for example, a WiMax (IEEE 802.16) network. A WLAN may be, for example, an IEEE 802.11x network. A WPAN may be, for example, a Bluetooth network, an IEEE 802.15x, or some other types of network. The techniques described herein may also be used for any combination of WAN, LAN, PAN, WWAN, WLAN, and/or WPAN. Communications subsystem 830 may permit data to be exchanged with a network, other computer systems, and/or any other devices. For example, communications subsystem 830 may be used to receive therapy determinations for therapeutic fluid (e.g., insulin) delivery, such as from a cloud computing system via an intermediary computing device (e.g., a controller) communicatively coupled to computing system 800, where processor(s) 810 may, based on the therapy determinations, send commands to an actuator controller to cause the delivery of appropriate amounts of therapeutic fluid (e.g., insulin) to a user. In another example, communications subsystem 830 may be used to communicate measurement results (e.g., sensor glucose levels) to a computing device (e.g., a smartphone or a personal health monitoring device) and/or to a remote server via the computing device, or receive data (e.g., calibration data, configuration data, etc.) from the computing device or the remote server via the computing device.

Computing system 800 may include a display module 850. Display module 850 may present information, such as text, images, audios, videos, and various instructions, from computing system 800 to a user. Such information may be derived from one or more application modules 822-824, communication/networking subsystem 130, one or more other hardware modules 870, a combination thereof, or any other suitable means. For example, display module 850 may be used to display user challenges that may include text, images, waveforms, audio clips, video clips, and the like. Display module 850 may use liquid crystal display (LCD) technology, light-emitting diode (LED) technology (including, for example, OLED, ILED, μLED, AMOLED, TOLED, etc.), light emitting polymer display (LPD) technology, or some other display technology.

Input/output user interface 860 may allow a user to send action requests to computing system 800 to perform particular actions, and may provide information (e.g., status of computing system 800, measurement results, alerts, etc.) to the user. Input/output user interface 860 may include one or more input devices, such as, for example, a touchscreen, a touch pad, microphone(s), button(s), dial(s), switch(es), a keyboard, a mouse, a game controller, or any other suitable device for receiving action requests and communicating the received action requests to processor(s) 810. In some embodiments, input/output user interface 860 may include one or more output devices, such as a display, a speaker, a light emitting device, a haptic device, and the like, to provide feedback or alarm to the user.

In some embodiments, computing system 800 may include a plurality of other hardware modules 870. Each of other hardware modules 870 may be a physical module within computing system 800. While each of other hardware modules 870 may be permanently configured as a structure, some of other hardware modules 870 may be temporarily configured to perform specific functions or temporarily activated. Examples of other hardware modules 870 may include, for example, an audio output and/or input module (e.g., a microphone or speaker), a near field communication (NFC) module, a rechargeable battery, a battery management system, a wired/wireless battery charging system, an actuator controller, and the like. In some embodiments, one or more functions of other hardware modules 870 may be implemented in software.

In various implementations, the above-described hardware and modules may be implemented on a single device or on multiple devices that can communicate with one another using wired or wireless connections. In alternative configurations, different and/or additional components may be included in computing system 800. Similarly, functionality of one or more of the components can be distributed among the components in a manner different from the manner described above.

Embodiments of the methods disclosed herein may be implemented by hardware, software, firmware, middleware, microcode, hardware description languages, or any combination thereof. For example, the operations may be performed by one or more servers or other computer systems. When implemented in software, firmware, middleware, or microcode, the program code or code segments to perform the associated tasks may be stored in one or more computer-readable media such as a storage medium, and may be executed by one or more processors to perform the associated tasks. The computer-readable media may include transitory or non-transitory computer-readable media, such as RAM, ROM, EEPROM, flash memory, solid-state drive, hard drive, CD, DVD, or any other medium that can be used to store desired program code in the form of instructions or data structures and that can be accessed by a computer. The one or more processors may include general purpose microprocessors, application specific integrated circuits (ASICs), graphic processing units (GPUs), network processing units (NPUs), digital signal processors (DSPs), field programmable logic arrays (FPGAs), and the like.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, systems, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing various embodiments. Various changes may be made in the function and arrangement of elements without departing from the spirit and scope of the present disclosure.

Also, some embodiments were described as processes depicted as flow diagrams or block diagrams. Although each may describe the operations as a sequential process, many of the operations may be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It will be apparent to those skilled in the art that substantial variations may be made in accordance with specific requirements. For example, customized or special-purpose hardware might also be used, and/or particular elements might be implemented in hardware, software (including portable software, such as applets, etc.), or both. Further, connection to other computing devices such as network input/output devices may be employed.

Any of the herein described techniques, operations, methods, programs, algorithms, or codes may be converted to, or expressed in, a programming language or computer program embodied on a computer, processor, or machine-readable medium. The terms “programming language” and “computer program,” as used herein, each include any language used to specify instructions to a computer or processor, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

With reference to the appended figures, components that can include memory can include non-transitory machine-readable media. The term “machine-readable medium” and “computer-readable medium” may refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media such as compact disk (CD) or digital versatile disk (DVD), punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code. A computer program product may include code and/or machine-executable instructions that may represent a procedure, a function, a subprogram, a program, a routine, an application (App), a subroutine, a module, a software package, a class, or any combination of instructions, data structures, or program statements.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that are also expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean A, B, C, or any combination of A, B, and/or C, such as AB, AC, BC, AA, ABC, AAB, AABBCCC, etc.

In this description, the recitation “based on” means “based at least in part on.” Therefore, if X is based on Y, then X may be a function of at least a part of Y and any number of other factors. If an action X is “based on” Y, then the action X may be based at least in part on at least a part of Y.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific embodiments have been described, these are not intended to be limiting. Various modifications and equivalents are within the scope of the following claims.

What is claimed is:

1. A processor-implemented method comprising:
obtaining data associated with a user and one or more medical devices of the user;
generating, based on the obtained data, insights related to the user's experience associated with the one or more medical devices;
determining personalized interventions for the user based on the insights; and
providing the personalized interventions through one or more communication channels or user interfaces.

2. The processor-implemented method of claim 1, wherein the insights include:
a patient experience score associated with the one or more medical devices;
factors that affect the patient experience score;
Shapley additive explanations (SHAP) values associated with the factors;
a priority of the factors;
a classification of the user; or
a combination thereof.

3. The processor-implemented method of claim 2, wherein the priority of the factors is determined based on the SHAP values associated with the factors.

4. The processor-implemented method of claim 1, wherein the data associated with the user and the one or more medical devices of the user comprises:
health condition data of the user;
operation data of the one or more medical devices;
the user's feedback associated with purchasing and/or using the one or more medical devices;
user interaction data; or
a combination thereof.

5. The processor-implemented method of claim 4, wherein:
the health condition data of the user includes glucose levels of the user; and
the operation data of the one or more medical devices includes insulin deliver data of an insulin delivery device.

6. The processor-implemented method of claim 4, wherein the user interaction data includes data associated with user interactions with the one or more medical devices, data associated with user interactions with a provider of the one or more medical devices, or a combination thereof.

7. The processor-implemented method of claim 1, wherein generating the insights comprises applying the obtained data to a machine learning model trained using historical data.

8. The processor-implemented method of claim 7, wherein the machine learning model includes an extreme gradient boosting (XGBoost) model trained to predict a patient experience score based on the obtained data.

9. The processor-implemented method of claim 1, further comprising:
extracting one or more features from the obtained data,
wherein generating the insights comprises applying the one or more features to an extreme gradient boosting (XGBoost) model.

10. The processor-implemented method of claim 1, wherein generating the insights comprises determining Shapley additive explanations (SHAP) values of one or more parameters of the obtained data for predicting a patient experience score.

11. The processor-implemented method of claim 1, wherein determining the personalized interventions for the user based on the insights comprises applying the insights to a large language model trained to generate the personalized interventions.

12. The processor-implemented method of claim 11, wherein the personalized interventions include:
proactive user interventions based on the insights;
reactive user interventions for troubleshooting;
personalized user education or training;
motivational coaching;
personalize care; or
a combination thereof.

13. The processor-implemented method of claim 1, wherein the one or more communication channels or user interfaces include:
one or more web applications;
one or more user applications;
one or more chatbots;
emails;
text messages;
one or more websites; or
a combination thereof.

14. A system comprising:
one or more processors; and
one or more processor-readable storage media storing instructions which, when executed by the one or more processors, cause performance of operations including:
obtaining data associated with a user and one or more medical devices of the user;
generating insights related to the user's experience associated with the one or more medical devices based on the obtained data;
determining personalized interventions for the user based on the insights and a large language model; and
providing the personalized interventions through one or more communication channels or user interfaces.

15. The system of claim 14, wherein generating the insights comprises:
applying the obtained data to an extreme gradient boosting (XGBoost) model trained using historical data to predict a patient experience score; and
determining Shapley additive explanations (SHAP) values of one or more parameters of the obtained data for predicting the patient experience score.

16. A medical device data management system comprising:
one or more data repositories configured to store collected data associated with a patient and one or more medical devices of the patient;
one or more insight generation engines configured to generate insights related to the patient's experience associated with the one or more medical devices based on the collected data;
an intervention engine configured to determine personalized interventions for the patient based on the insights; and
one or more communication interfaces configured to provide the personalized interventions to one or more users of the medical device data management system.

17. The medical device data management system of claim 16, wherein the insights include:
a patient experience score associated with the one or more medical devices;
factors that affect the patient experience score;
Shapley additive explanations (SHAP) values associated with the factors;
a priority of the factors;
a classification of the patient; or
a combination thereof.

18. The medical device data management system of claim 16, wherein the data associated with the patient and the one or more medical devices of the patient comprises:
health condition data of the patient;
operation data of the one or more medical devices;
the patient's feedback associated with purchasing and/or using the one or more medical devices;
user interaction data; or
a combination thereof.

19. The medical device data management system of claim 16, wherein the one or more insight generation engines are configured to:
extract one or more features from the collected data;
apply at least one of the collected data or the one or more features to an extreme gradient boosting (XGBoost) model trained using historical data to predict a patient experience score;
determine Shapley additive explanations (SHAP) values of one or more parameters for predicting the patient experience score; or
a combination thereof.

20. The medical device data management system of claim 16, wherein the intervention engine is configured to apply the insights to a large language model trained to generate the personalized interventions, the personalized interventions including:
proactive user interventions based on the insights;
reactive user interventions for troubleshooting;
personalized user education or training;
motivational coaching;
personalize care; or
a combination thereof.
